(19) United States
(12) Patent Application Publication
(10) Pub. No.: US 2019/0024184 A1
(43) Pub. Date: Jan. 24, 2019

(54) DISTINGUISHING METASTATIC-LETHAL PROSTATE CANCER FROM INDOLENT PROSTATE CANCER USING METHYLATION STATUS OF EPIGENETIC MARKERS

(71) Applicant: Fred Hutchinson Cancer Research Center, Seattle, WA (US)

(72) Inventors: Janet Stanford, Seattle, WA (US); Ziding Feng, Seattle, WA (US)

(73) Assignee: Fred Hutchinson Cancer Research Center, Seattle, WA (US)

(21) Appl. No.: 16/065,662

(22) PCT Filed: Dec. 22, 2016
(86) PCT No.: PCT/US2016/068291

§ 371 Date: Jun. 22, 2018

Related U.S. Application Data
(60) Provisional application No. 62/387,273, filed on Dec. 23, 2015.

(51) Int. Cl. C12Q 1/6886

(52) U.S. Cl. CPC C12Q 1/6886; C12Q 2600/154

(57) ABSTRACT

Methods and kits to distinguish metastatic-lethal prostate cancer (PCa) from indolent PCa in a subject are described. The methods and kits utilize the methylation status of genetic markers. Distinguishing metastatic-lethal PCa from indolent PCa informs more directed treatments at an earlier time point than previously available.

### CROSS REFERENCE TO RELATED APPLICATION

This application claims priority to U.S. Provisional Patent Application No. 62/387,273 filed on Dec. 23, 2015, which is incorporated by reference herein in its entirety.

### STATEMENT REGARDING FEDERALLY SPONSORED RESEARCH OR DEVELOPMENT

This invention was made with government support under CA097186 awarded by the National Institutes of Health. The government has certain rights in the invention.

### REFERENCE TO SEQUENCE LISTING

A computer readable text file, entitled “DN1LK5845.txt (Sequence Listing.txt)” created on or about Dec. 20, 2016, with a file size of 612 KB, contains the sequence listing for this application and is hereby incorporated by reference in its entirety.

### FIELD OF THE DISCLOSURE

The present disclosure provides methods and kits to distinguish metastatic-lethal prostate cancer (PCa) from indolent PCa using the methylation status of epigenetic markers evaluated in tumor tissue. The epigenetic markers include CpG methylation sites within Intergenic 1, Intergenic 2, Intergenic 3, PI15, FHAD1, ALKBH5, KLHL8, and/or ATP11A.

### BACKGROUND OF THE DISCLOSURE

Prostate cancer (PCa) is a biologically and clinically heterogeneous disease with 220,800 new cases and 27,540 cancer-specific deaths expected in the U.S. for 2015 and over 300,000 deaths worldwide each year. PCa most often has an indolent course, but a subset of 20-30% of patients progress to metastasis and eventually die from PCa. To date, the single most important predictor of prognosis is the Gleason score, which a pathologist assigns by histologic examination of biopsy tissue. However, Gleason score is frequently inaccurate, particularly when a small amount of tumor is available. By comparing the Gleason score of the diagnostic biopsy to subsequent prostatectomy, upgrading and downgrading occurs in 14% to 51% and 9%, respectively. Furthermore, while a tumor with Gleason score 3+3=6 is low-risk and Gleason score 4+4=8 or greater is high-risk, tumors that are 3+4 or 4+3 are heterogeneous and include a substantial proportion of tumors. Thus, research efforts are focused on finding prognostic biomarkers that can improve patient classification for targeting therapies to those patients most likely to benefit.

Recent biomarker studies have mostly focused on altered tumor tissue gene expression profiles, leading to development of tests for mRNA signatures of tumor aggressiveness. Epigenetic alterations in tumor DNA may also provide valuable prognostic information. The most widely studied epigenetic modification is DNA methylation, which occurs at CpG sites across the genome and regulates gene expression. To date, studies of DNA methylation and PCa progression have been limited to small sets of candidate genes in relation to biochemical (i.e., prostate-specific antigen, PSA) recurrence. Although patients with biochemical progression are at higher risk of PCa-related death, this is still a biologically heterogeneous group and most will not die from PCa. Studies of patients with biochemical recurrence after radical prostatectomy found that only 17% to 21.5% died of PCa after a median follow-up of 10 years.

### SUMMARY OF THE DISCLOSURE

Epigenome-wide DNA methylation profiles in primary prostate cancers were investigated. The study included a population-based radical prostatectomy cohort with long-term follow-up for metastatic progression and cancer-specific mortality. The goal of this study was to detect differentially methylated CpG biomarkers that distinguish metastatic-lethal PCa from indolent PCa or non-recurrent disease.

Eight differentially methylated CpGs that distinguish tumors with metastatic-lethal potential from more indolent tumors that do not recur within five or more years after diagnosis were identified and then validated in an independent patient cohort. These epigenetic biomarkers improve prognostic determination and clinical decision making for newly diagnosed prostate cancer patients with clinically localized disease. These CpG markers include Intergenic 1; Intergenic 2; FHAD1; ALKBH5; KLHL8; ATP11A; Intergenic 3; and PI15. Combination of detection of methylations status of these CpG markers improves the prognostic discrimination of Gleason scores.

### BRIEF DESCRIPTION OF THE FIGURES

FIG. 1. Characteristics of the two Prostate Cancer (PCa) patient populations.

FIG. 2. Top-ranked 42 DNA methylation biomarkers for classification of metastatic-lethal PCa in the Fred Hutchinson Cancer Research Center (FH) cohort.

FIG. 3. Number of times the 42 DNA methylation biomarkers were selected in the bootstrap samples (n=100, per Criterion).

FIG. 4. Eight validated DNA methylation biomarkers for classification of metastatic-lethal PCa in the Eastern Virginia Medical School (EV) cohort.

FIG. 5. ROC curves for predicting metastatic-lethal PCa for eight validated DNA methylation biomarkers and Gleason score.

FIG. 6. Predictive performance of eight validated DNA methylation biomarkers for distinguishing metastatic-lethal PCa in models combining each CpG with Gleason score in the EV cohort.

FIG. 7. Primers used for pyrosequencing. All primers are listed in the 5′-3′ orientation.

FIG. 8. Correlation of pyrosequencing results with HM450 results.

FIGS. 9A, 9B. (9A) Selection model building using the 5 CpG markers validated by pyrosequencing. (9B) Fitted model results using the 5 CpG markers with Gleason score, compared to Gleason score alone.

### DETAILED DESCRIPTION

Prostate cancer (PCa) is a biologically and clinically heterogeneous disease with 220,800 new cases and 27,540 cancer-specific deaths expected in the U.S. for 2015 and over 300,000 deaths worldwide each year. PCa can be indolent (i.e., does not recur within 5 years of diagnosis and treatment, and may not cause problems even if left untreated) or can recur. These types of PCa are distinguished from metastatic-lethal PCa.

Currently, the single most important predictor of prognosis is Gleason score, which a pathologist assigns by histologic examination of biopsy tissue. However, Gleason score is frequently inaccurate, particularly when a small amount of tumor is available. By comparing the Gleason score of the diagnostic biopsy to subsequent prostatectomy, upgrading and downgrading occurs in 14% to 51% and 9%, respectively. Furthermore, while a tumor with Gleason score 3+3=6 is low-risk and Gleason score 4+4=8 or greater is high-risk, tumors that are 3+4 or 4+3 are heterogeneous and include the majority of tumors. Thus, research efforts are focused on finding prognostic biomarkers that can improve stratification of metastatic-lethal PCa from indolent PCa for targeting therapies to those patients most likely to benefit.

Recent biomarker studies have mostly focused on altered gene expression, leading to development of tests for mRNA signatures of tumor aggressiveness. Epigenetic alterations in tumor DNA may also provide valuable prognostic information. Epigenetics refers to changes in gene expression that are not due to mutations (i.e. changes in the sequence, such as loss or gain of nucleotides, of a gene). Thus, epigenetics is a reversible regulation of gene expression caused by several mechanisms other than mutation.

The most widely studied epigenetic modification is DNA methylation. Other epigenetic changes include changes to the three dimensional structure of DNA, histone protein modification, micro-RNA inhibitory activity, imprinting, X-inactivation, and long-distance chromosomal interaction.

DNA methylation occurs at CpG sites across the genome and regulates gene expression. Cytosine is one of a group of four building blocks (i.e., nucleotides) from which DNA is constructed (i.e. cytosine (C), thiamine (T), adenine (A), and guanosine (G)). The chemical structure of cytosine is in the form of a six-sided hexagon or pyrimidine ring. Cytosine can be paired with guanosine in a linear sequence along the single DNA strand to form 5′-CG-3′, or CpG pairs. “CpG” refers to a cytosine-phosphate-guanosine chemical bond in which the phosphate binds the two nucleotides together. In mammals, in 70-80% of these CpG pairs the cytosine is methylated. (Chatterjee, et al., Biochemica et Biophisica Acta 2012; 1819:763-70).

The term “CpG island” refers to regions in the genome with a high concentration of CG dinucleotide pairs or CpG sites. The length of DNA occupied by the CpG island is usually 300-3000 base pairs. The CpG island can be defined by various criteria including the length of recurrent CG dinucleotide pairs occupying at least 200 base pair (bp) of DNA, a CG content of the segment of at least 50%, and/or that the observed/expected CpG ratio is greater than 60%. There are an estimated 28-30 million CpG sites across the genome.

CpG islands are commonly found in gene promoters. Across mammals, an average of forty percent of gene promoters contain CpG islands (Fatemi, et al., Nucleic Acids Res. 2005; 33:e176). Gene promoters are particularly CG-rich in the human genome, as 70% of promoters in the human genome have high CG content. Although CpG islands are highly associated with gene promoters, CpG islands can also exist in other regions of the genome (such as in gene bodies or in intergenic regions).

In most CpG sites scattered throughout the DNA the cytosine nucleotide is methylated. In contrast, the cytosine is more often unmethylated in CpG sites located in the CpG islands of the promoter regions of genes, supporting a role of methylation status of cytosine in CpG islands in gene transcriptional activity.

Methylation of cytosine refers to the enzymatic addition of a methyl group or single carbon atom to position #5 of the pyrimidine ring of cytosine, which leads to the conversion of cytosine to 5-methyl-cytosine. The methylation of cytosine can be accomplished by a family of enzymes called DNA methyltransferases (DNMTs). The 5-methyl-cytosine, when formed, is prone to mutation or the chemical transformation of the original cytosine to form thymine. Five-methyl-cytosines account for 1% of the nucleotide bases overall in the normal human genome.

As indicated previously, the methylation status of cytosine throughout the DNA can be said to indirectly indicate the relative expression status of multiple genes throughout the genome. The methylation of cytosine nucleotides within a gene, particularly in the promoter region of the gene, is known to be a mechanism of controlling overall gene activity, i.e. mRNA and protein synthesis. Classically, the methylation of cytosine is associated with inhibition of gene transcription. However, in certain genes, methylation of cytosine is known to have the reverse effect and instead promotes gene transcription.

To date, studies of DNA methylation and PCa progression have been limited to small sets of candidate genes in relation to biochemical recurrence. Biochemical recurrence can be determined by measurement of the level of a PCa marker (i.e. prostate-specific antigen, PSA) in a patient's blood sample, wherein a PSA level above a certain threshold can indicate cancer recurrence. Hypermethylation of CpGs in the promoter region of PITX2 and GSTP1 was associated with PSA recurrence. Although patients with biochemical progression are at higher risk of PCa death, this is still a biologically heterogeneous group and most will not die from PCa. More particularly, studies of patients with biochemical recurrence after radical prostatectomy found that only 17% to 21.5% died of PCa after a median follow-up of 10 years.

For the current disclosure, epigenome-wide DNA methylation profiles in primary PCa were investigated. The study included a population-based radical prostatectomy cohort with long-term follow-up for metastatic progression and cancer-specific mortality. The goal of the disclosed study was to detect differentially methylated CpG biomarkers that distinguish patients with metastatic-lethal PCa from those with indolent, non-recurrent disease. The most robust methylation biomarkers identified were then tested in a validation cohort.

Strengths of the current study include its larger sample size, the epigenome-wide approach for biomarker discovery, and the population-based nature of the discovery cohort, with long-term follow-up of patients diagnosed with and treated for clinically localized PCa. Validation of the DNA methylation biomarkers in an independent patient cohort is also critical, and confirms that these CpGs have added value to Gleason score for predicting adverse outcomes.

The results described herein demonstrate that detecting DNA methylation biomarkers in primary tumor tissue obtained at surgical resection can distinguish metastatic-lethal PCa patients from those men at least five years post-radical prostatectomy without recurrence. Of the 42 top-ranked differentially methylated CpG sites that stratified patients with aggressive tumors in the discovery cohort, and improved the prognostic discrimination beyond that provided by Gleason score alone, eight were subsequently validated in an independent patient cohort.

The eight differentially methylated CpG sites validated in this study for the metastatic-lethal tumor phenotype are located in five genes (ALKBH5, ATP11A, FHAD1, KLHL8, and PI15) and three intergenic regions (referred to herein as “Intergenic 1”; “Intergenic 2” and “Intergenic 3”). The five genes are involved in regulatory functions, response to hypoxia, protein-binding, developmental processes, and ion transport. Dhanoa et al., Hum Genomics 7:13, 2013; Durocher et al., FEBS Lett 513:58-66, 2002; Falak et al., Physiol Genomics 46:418-28, 2014; Miyoshi et al., Oncol Rep 23:505-10, 2010; Thalhammer et al., PLoS One 6:e16210, 2011. The oxidative DNA demethylase ALKBH5, which is upregulated under hypoxia and also plays a role in spermatogenesis, belongs to the same gene family as ALKBH3 (PCa Antigen 1), which is highly expressed in prostate tumors and is a potential therapeutic target for PCa. Koike et al., Curr Cancer Drug Targets 12:847-56, 2012. In a previous study expression of ATP11A, which belongs to an extended family of adenosine triphosphate-binding cassette transporters, was associated with colorectal cancer death. Miyoshi et al., Oncol Rep 23:505-10, 2010. Another previous study found that PI15 (peptidase inhibitor 15) was amplified and overexpressed in some advanced prostate tumors. Vainio et al., Prostate 72:789-802, 2012. Aberrant DNA methylation of PI15 and ATP11A correlated with mRNA expression of these genes in the same patients' tumors. For PI15, the correlation was in the expected direction (i.e., promoter hypermethylation and reduced expression).

The methods and kits utilize differential methylation to distinguish metastatic-lethal PCa from indolent PCa. Metastatic-lethal PCa recurs, metastasizes and subsequently leads to death from PCa after initial diagnosis. Indolent PCa does not recur or relapse within 5 years of diagnosis and treatment.

In particular embodiments, differential methylation between metastatic-lethal PCa and indolent PCa is detected in one or more of: Intergenic 1 (e.g., CpG site: cg01135464); Intergenic 2 (e.g., CpG site: cg02223001); FHAD1 (e.g., CpG site: cg02394978); ALKBH5 (e.g., CpG site: cg07166550); KLHL8 (e.g., CpG site: cg16713292); ATP11A (e.g., CpG site: cg21513610); Intergenic 3 (e.g., CpG site: cg22501793); and PI15 (e.g., CpG site: cg24349665). In particular embodiments, differential methylation between metastatic-lethal PCa and indolent PCa is detected in all CpG sites within the disclosed genes and/or intergenic regions.

Particular embodiments detect differential methylation of Intergenic 1; Intergenic 2; FHAD1; ALKBH5; KLHL8; ATP11A; Intergenic 3; and PI15.

Particular embodiments detect differential methylation of Intergenic 1 CpG sites in combination with 1, 2, 3, 4, 5, 6 or 7 markers selected from Intergenic 2 CpG sites; FHAD1 CpG sites; ALKBH5 CpG sites; KLHL8 CpG sites; ATP11A CpG sites; Intergenic 3 CpG sites; and PI15 CpG sites.

Particular embodiments detect differential methylation of Intergenic 2 CpG sites in combination with 1, 2, 3, 4, 5, 6 or 7 markers selected from Intergenic 1 CpG sites; FHAD1 CpG sites; ALKBH5 CpG sites; KLHL8 CpG sites; ATP11A CpG sites; Intergenic 3 CpG sites; and PI15 CpG sites.

Particular embodiments detect differential methylation of FHAD1 CpG sites in combination with 1, 2, 3, 4, 5, 6 or 7 markers selected from Intergenic 1 CpG sites; Intergenic 2 CpG sites; ALKBH5 CpG sites; KLHL8 CpG sites; ATP11A CpG sites; Intergenic 3 CpG sites; and PI15 CpG sites.

Particular embodiments detect differential methylation of ALKBH5 CpG sites in combination with 1, 2, 3, 4, 5, 6 or 7 markers selected from Intergenic 1 CpG sites; Intergenic 2 CpG sites; FHAD1 CpG sites; KLHL8 CpG sites; ATP11A CpG sites; Intergenic 3 CpG sites; and PI15 CpG sites.

Particular embodiments detect differential methylation of KLHL8 CpG sites in combination with 1, 2, 3, 4, 5, 6 or 7 markers selected from Intergenic 1 CpG sites; Intergenic 2 CpG sites; FHAD1 CpG sites; ALKBH5 CpG sites; ATP11A CpG sites; Intergenic 3 CpG sites; and PI15 CpG sites.

Particular embodiments detect differential methylation of ATP11A CpG sites in combination with 1, 2, 3, 4, 5, 6 or 7 markers selected from Intergenic 1 CpG sites; Intergenic 2 CpG sites; FHAD1 CpG sites; ALKBH5 CpG sites; KLHL8 CpG sites; Intergenic 3 CpG sites; and PI15 CpG sites.

Particular embodiments detect differential methylation of Intergenic 3 CpG sites in combination with 1, 2, 3, 4, 5, 6 or 7 markers selected from Intergenic 1 CpG sites; Intergenic 2 CpG sites; FHAD1 CpG sites; ALKBH5 CpG sites; KLHL8 CpG sites; ATP11A CpG sites; and PI15 CpG sites.

Particular embodiments detect differential methylation of PI15 CpG sites in combination with 1, 2, 3, 4, 5, 6 or 7 markers selected from Intergenic 1 CpG sites; Intergenic 2 CpG sites; FHAD1 CpG sites; ALKBH5 CpG sites; KLHL8 CpG sites; ATP11A CpG sites; and Intergenic 3 CpG sites.

Particular embodiments can also exclude particular markers. For example, in particular embodiments, Intergenic 1 CpG sites are excluded. In particular embodiments, Intergenic 2 CpG sites are excluded. In particular embodiments, FHAD1 CpG sites are excluded. In particular embodiments, ALKBH5 CpG sites are excluded. In particular embodiments, KLHL8 CpG sites are excluded. In particular embodiments, ATP11A CpG sites are excluded. In particular embodiments, Intergenic 3 CpG sites are excluded. In particular embodiments, PI15 CpG sites are excluded. In particular embodiments, more than one marker is excluded, two or more markers are excluded, three or more markers are excluded, four or markers are excluded, five or more markers are excluded, 6 or more markers are excluded or 7 markers are excluded.

In particular embodiments when more than one marker is assayed, values of the detected markers can be calculated into a score. Each value can be weighted evenly within an algorithm generating a score, or the values for particular markers can be weighted more heavily in reaching the score. For example, markers with higher AUC or pAUC and/or methylation difference scores could be weighted more heavily than markers with lower AUC or pAUC and/or methylation difference scores. For example, in particular embodiments, Intergenic 1, KLHL8, and/or ATP11A may be weighted more heavily than other markers in a panel.

Markers may also be grouped into classes, and each class given a weighted score. For example, marker values for distinguishing metastatic-lethal PCa from indolent PCa may be grouped into classes and weighted as follows (from highest weight to lowest weight): Class 1: Intergenic 1, KLHL8, and ATP11A; Class 2: PI15 and FHAD1; and Class 3: Intergenic 2, ALKBH5, and Intergenic 3.

Any marker or class of markers can be included in a particular value calculation. For example, in particular embodiments, Class 1 is included. In particular embodiments, Class 2 is included. In particular embodiments, Class 3 is included. In particular embodiments, groups of classes can be included, for example, Classes 1 and 2; 1 and 3; and/or 2 and 3. Particular classes can also be excluded. For example, in particular embodiments, Class 1 is excluded. In particular embodiments, Class 2 is excluded. In particular embodiments, Class 3 is excluded.

Up (hyper)- or down (hypo)-methylation of the markers (e.g., methylation status) can be assessed by detecting methylation status and comparing a value to a relevant reference level. For example, the methylation status of one or more markers can be indicated as a value. The value can be one or more numerical values resulting from the assaying of a sample, and can be derived, e.g., by measuring methylation status of the marker(s) in the sample by an assay, or from a dataset obtained from a provider such as a laboratory, or from a dataset stored on a server.

In the broadest sense, the value may be qualitative or quantitative. As such, where detection is qualitative, the methods and kits provide a reading or evaluation, e.g., assessment, of whether or not the marker is methylated in the sample being assayed. In further embodiments, the methods and kits provide a quantitative detection of methylation, i.e., an evaluation or assessment of the actual amount or relative abundance of methylation of the marker in the sample being assayed. In such embodiments, the quantitative detection may be absolute or relative, if the method is a method of detecting methylation of two or more different markers in a sample. As such, the term “quantifying” when used in the context of quantifying methylation of a marker in a sample can refer to absolute or to relative quantification. Absolute quantification can be accomplished by inclusion of samples with known methylation parameters as one or more control markers and referencing, e.g., normalizing, the detected methylation level of the experimental marker with the known control markers (e.g., through generation of a standard curve). Alternatively, relative quantification can be accomplished by comparison of detected methylation levels or amounts between two or more different markers to provide a relative quantification of each of the two or more markers, e.g., relative to each other. The actual measurement of values for the markers can be determined using any method known in the art.

As stated previously, detected marker levels (e.g., values) can be compared to one or more reference levels. Reference levels can be obtained from one or more relevant datasets. A “dataset” as used herein is a set of numerical values resulting from evaluation of a sample (or population of samples) under a desired condition. The values of the dataset can be obtained, for example, by experimentally obtaining measures from sample(s) and constructing a dataset from these measurements. As is understood by one of ordinary skill in the art, the reference level can be based on e.g., any mathematical or statistical formula useful and known in the art for arriving at a meaningful aggregate reference level from a collection of individual datapoints; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a population. A “population” is any grouping of subjects or samples of like specified characteristics. The grouping could be according to, for example, clinical parameters, clinical assessments, therapeutic regimens, disease status, severity of PCa, etc. In particular embodiments, a population is a group of subjects with metastatic-lethal PCa. In particular embodiments, a population is a group of subjects with indolent PCa.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than 0.05.

In particular embodiments, values obtained based on the markers and/or other dataset components can be subjected to an analytic process with chosen parameters. The parameters of the analytic process may be those disclosed herein or those derived using the guidelines described herein. The analytic process used to generate a result may be any type of process capable of detecting indolent or metastatic-lethal PCa based on methylation status detection, for example, a linear algorithm, a quadratic algorithm, a decision tree algorithm, or a voting algorithm. The analytic process may set a threshold for determining the probability that a sample belongs to a given class. The probability preferably is at least 60%, at least 70%, at least 80%, at least 90%, at least 95% or higher. Detection relies on performing an assay on a biological sample, such as a primary PCa tumor sample.

The receiver operating characteristics (ROC) curve is a graph plotting sensitivity (true positive rate), which is defined in this setting as the percentage of metastatic-lethal PCa cases with a positive test or abnormal (differential) cytosine methylation level at a particular cytosine locus on the Y axis and false positive rate (1-specificity), i.e. the number of indolent (non-recurrent) PCa cases with abnormal (differential) cytosine methylation at the same locus on the X-axis. Specificity is defined as the percentage of normal (i.e., non-recurrent) cases with normal methylation levels at the locus of interest or a negative test. False positive rate refers to the percentage of normal (e.g., non-recurrent) subjects falsely found to have a positive test (i.e. abnormal or differential methylation levels).

The area under the ROC curves (AUC) indicates the accuracy of the test in identifying normal from abnormal cases (Hanley & McNeil, Radiology 1982; 143:29-36). The AUC is the area under the ROC plot from the curve to the diagonal line from the point of intersection of the X- and Y-axes and with an angle of incline of 45° (a test with no discrimination between two groups, e.g., metastatic-lethal vs. non-recurrent PCa cases, has a 45° diagonal line from the lower left to the upper right corner). The higher the area under the receiver operating characteristics (ROC) curve, the greater the accuracy of the test in predicting the condition of interest. An area ROC=1.0 indicates a perfect test, which is positive in all cases with the disorder (e.g, metastatic-lethal PCa) and negative in all normal individuals without the disorder (e.g., non-recurrent PCa). Thus, the closer the plot is to the upper left corner, the higher the overall accuracy of the test.

Methylation of the markers can be assessed using various methylation detection assays. A “methylation detection assay” refers to an assay, which can be commercially available, for distinguishing methylated versus unmethylated cytosine loci in DNA. Techniques for measuring cytosine methylation include bisulfite-based methylation assays. The addition of bisulfite to DNA results in the methylation of the unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

Quantitative methylation detection assays include combined bisulfite and restriction analysis COBRA, which uses methylation sensitive restriction endonuclease, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay is performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight™ (Qiagen, Redwood City, Calif.) a quantitative methylation detection assay that uses fluorescence based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are used to amplify the target sequence of interest. The amplified PCR product is isolated and used to quantitate the methylation status of the CpG site of interest. (Gonzalgo and Jones Nuclei Acids Res 1997; 25:252-31).

In particular embodiments, the Infinium® (Ilumina, Inc., San Diego Calif., USA) Human Methylation 450 Beadchip assay is used. The Ilumina assay can be used for genome wide quantitative methylation profiling. In particular embodiments, genomic DNA can be extracted from cells. Genomic DNA can be isolated and proteins or other contaminants can be removed from the DNA using proteinase K. The DNA can then be removed from the solution using available methods such as organic extraction, salting out, or binding the DNA to a solid phase support. As described above, and in the Infinium® Assay Methylation Protocol Guide, the DNA can be treated with sodium bisulfite, which converts unmethylated cytosine to uracil, while the methylated cytosine remains unchanged. The bisulfite converted DNA can then be denatured and neutralized. The denatured DNA can then be amplified. The next step uses enzymatic means to fragment the DNA. The fragmented DNA can then be precipitated using isopropanol and separated by centrifugation. The separated DNA can next be suspended in a hybridization buffer. The fragmented DNA can then be hybridized to beads that have been covalently limited to 50mer nucleotide segments at a locus specific to the cytosine nucleotide of interest in the genome. There are a total of over 500,000 bead types specifically designed to anneal to the locus where the particular cytosine is located. The beads are bound to silicon based arrays. There are two bead types designed for each locus, one bead type represents a probe that is designed to match to the methylated locus at which the cytosine nucleotide will remain unchanged. The other bead type corresponds to an initially unmethylated cytosine, which after sodium bisulfite treatment, is converted to uracil and ultimately a thiamine nucleotide. Unhybridized DNA (DNA not annealed to the beads) is washed away leaving only DNA segments bound to the appropriate bead and containing the cytosine of interest. If the cytosine of interest was unmethylated prior to the sodium bisulfite treatment, then it will match with the unmethylated or “U” bead probe. This enables single base extensions with fluorescent labeled nucleotide probes and generate fluorescent signals for that bead probe that can be read in an automated fashion. If the cytosine is methylated, single base mismatch will occur with the “U” bead probe oligomer. No further nucleotide extension on the bead oligomer occurs, thus preventing incorporation of the fluorescent tagged nucleotides on the bead. This will lead to low fluorescent signal from the “U” bead. The reverse will happen on the “M” or methylated bead probe.

Lasers are then used to stimulate the fluorophore bound to the single-base used for the sequence extension. The level of methylation at each cytosine locus is detected by the intensity of the fluorescence from the methylated compared to the unmethylated bead. Cytosine methylation level is expressed as “p” which is the ratio of the methylated-bead probe signal to total signal intensity at that cytosine locus.

In particular embodiments, reliable identification of specific cytosine loci distributed throughout the genome has been detailed in, for example, the document “CpG Loci Identification. A guide to Ilumina's method for unambiguous CpG loci identification and tracking for the GoldenGate® and Infinium® assays for Methylation”. Briefly, Illumina has developed a CpG locus identifier that designates cytosine loci based on the actual or contextual sequence of nucleotides in which the cytosine is located. It uses a similar strategy as used by NCBI's re SNP IPS (rs#) and is based on the sequence flanking the cytosine of interest. Thus a unique CpG locus cluster ID number is assigned to each of the cytosine undergoing evaluation. The system is consistent and not affected by changes in public databases and genome assemblies. Flanking sequences of 60 bases 5′ and 3′ to the CG locus (i.e. a total of 122 base sequences) is used to identify the locus. Thus a unique “CpG cluster number” or cg# is assigned to the sequence of 122 bp which contains the CpG of interest. Thus, only if the 122 bp in the CpG cluster is identical is there a risk of a locus being assigned the same number and being located in more than one position in the genome. Three separate criteria are utilized to track an individual CpG locus based on this unique ID system, chromosome number, genomic coordinate, and genome build. The lesser of the two coordinates “C” or “G” in CpG is used in the unique CG loci identification. The CG locus is also designated in relation to the first “unambiguous” pair of nucleotides containing either an ‘A’ or ‘T’. If one of these nucleotides is 5′ to the CG then the arrangement is designated TOP and if such a nucleotide is 3′ it is designated BOT.

In particular embodiments, pyrosequencing is used to detect marker methylation. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, which converts unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al. Methods Mol Biol. 2015 1343: 249-264. Pyrosequencing is especially useful for detecting methylation in the CpG sites within genes.

In addition, the forward or reverse DNA strand is indicated as being the location of the cytosine being evaluated. The assumption is made that methylation status of cytosine bases within the specific chromosome region is synchronized (Eckhart, et al., Nat. Gent. 2006, 38:1379-85).

Measurement of mRNA levels transcribed by genes with altered cytosine methylation can also be assessed. Any technique for determining expression levels of mRNA can be used including Northern blot analysis, fluorescent in situ hybridization (FISH), RNase protection assays (RPA), microarrays, PCR-based, or other technologies for measuring RNA levels can be used.

Up (hyper)- or down (hypo)-methylation of genes also can be detected indirectly using, for example, cDNA arrays, cDNA fragment fingerprinting, cDNA sequencing, clone hybridization, differential display, differential screening, FRET detection, liquid microarrays, PCR, RT-PCR, quantitative RT-PCR analysis with TaqMan assays, molecular beacons, microelectric arrays, oligonucleotide arrays, polynucleotide arrays, serial analysis of gene expression (SAGE), and/or subtractive hybridization.

Further hybridization technologies that may be used are described in, for example, U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; and 5,800,992 as well as WO 95/21265; WO 96/31622; WO 97/10365; WO 97/27317; EP 373 203; and EP 785 280.

Additionally, protein products of genes that are differentially methylated can be measured to indirectly assess cytosine methylation levels. Proteins translated from mRNA reflect the same phenomenon of altered gene expression related to changes in cytosine methylation. Therefore, protein expression could also be used to biologically classify a sample as metastatic-lethal or indolent PCa.

“Protein detection” includes detection of full-length proteins, mature proteins, pre-proteins, polypeptides, isoforms, mutations, post-translationally modified proteins and variants thereof, and can be detected in any suitable manner.

In particular embodiments, a protein marker is detected by contacting a sample with reagents (e.g., antibodies), generating complexes of reagent and marker(s), and detecting the complexes. Particular embodiments for detecting and measuring protein levels can use methods including agglutination, chemiluminescence, electro-chemiluminescence (ECL), enzyme-linked immunoassays (ELISA), immunoassay, immunoblotting, immunodiffusion, immunoelectrophoresis, immunofluorescence, immunohistochemistry, immunoprecipitation, mass-spectrometry, and western blot. See also, e.g., E. Maggio, Enzyme-Immunoassay (1980), CRC Press, Inc., Boca Raton, Fla.; and U.S. Pat. Nos. 4,727,022; 4,659,678; 4,376,110; 4,275,149; 4,233,402; and 4,230,797.

Nucleic acids and proteins can be linked to chips, such as microarray chips. See, for example, U.S. Pat. Nos. 5,143,854; 6,087,112; 5,215,882; 5,707,807; 5,807,522; 5,958,342; 5,994,076; 6,004,755; 6,048,695; 6,060,240; 6,090,556; and 6,040,138. Binding to nucleic acids or proteins on microarrays can be detected by scanning the microarray with a variety of laser or charge coupled device (CCD)-based scanners, and extracting features with software packages, for example, Imagene (Biodiscovery, Hawthorne, Calif.), Feature Extraction Software (Agilent), Scanalyze (Eisen, M. 1999. SCANALYZE User Manual; Stanford Univ., Stanford, Calif. Ver 2.32.), or GenePix (Axon Instruments).

Embodiments disclosed herein can be used with high throughput screening (HTS). Typically, HTS refers to a format that performs at least 100 assays, at least 500 assays, at least 1000 assays, at least 5000 assays, at least 10,000 assays, or more per day. When enumerating assays, either the number of samples or the number of protein or nucleic acid markers assayed can be considered.

Generally, HTS methods involve a logical or physical array of either samples, or the nucleic acid or protein markers, or both. Appropriate array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384, or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis.

HTS assays and screening systems are commercially available from, for example, Zymark Corp. (Hopkinton, Mass.); Air Technical Industries (Mentor, Ohio); Beckman Instruments, Inc. (Fullerton, Calif.); Precision Systems, Inc. (Natick, Mass.), etc. These systems typically automate entire procedures including all sample and reagent pipetting, liquid dispensing, timed incubations, and final readings of the microplate in detector(s) appropriate for the assay. These configurable systems provide HTS as well as a high degree of flexibility and customization. The manufacturers of such systems provide detailed protocols for the various methods of HTS.

Disclosed kits include materials and reagents necessary to assay a sample for the methylation status of one or more markers disclosed herein. The materials and reagents can include those necessary to assay the markers disclosed herein according to any method described herein and/or known to one of ordinary skill in the art.

Various embodiments include materials and reagents necessary to perform methylation detection assays on particular epigenetic loci. Particular embodiments include materials and reagents necessary to assay for up- or down-methylation of a marker protein in a sample. In particular embodiments, the kits include antibodies to marker proteins and/or can also include aptamers (oligonucleotides or peptides that bind specific molecules), epitopes (regions of antigens recognized by an antibody, BCR or TCR), or mimitopes (molecules designed to mimic the binding properties of an epitope). Other embodiments additionally or alternatively include oligonucleotides that specifically assay for one or more marker nucleic acids based on homology and/or complementarity with marker nucleic acids. The oligonucleotide sequences may correspond to fragments of the marker nucleic acids. For example, the oligonucleotides can be more than 200, 175, 150, 100, 50, 25, 10, or fewer than 10 nucleotides in length. Collectively, any molecule (e.g., antibody, aptamer, epitope, mimitope, oligonucleotide) that forms a complex with a marker can be referred to as a marker binding agent herein.

Embodiments of kits can contain in separate containers marker binding agents either bound to a matrix, or packaged separately with reagents for binding to a matrix. In particular embodiments, the matrix is, for example, a porous strip. In particular embodiments, measurement or detection regions of the porous strip can include a plurality of sites containing marker binding agents. In particular embodiments, the porous strip can also contain sites for negative and/or positive controls. Alternatively, control sites can be located on a separate strip from the porous strip. Optionally, the different detection sites can contain different amounts of marker binding agents, e.g., a higher amount in the first detection site and lesser amounts in subsequent sites. Upon the addition of test sample, the number of sites displaying a detectable signal provides a quantitative indication of the amount of marker present in the sample. The detection sites can be configured in any suitably detectable shape and can be, e.g., in the shape of a bar or dot spanning the width (or a portion thereof) of a porous strip.

In particular embodiments the matrix can be a solid substrate, such as a “chip.” See, e.g., U.S. Pat. No. 5,744,305. In particular embodiments the matrix can be a solution array; e.g., xMAP (Luminex, Austin, Tex.), Cyvera (Illumina, San Diego, Calif.), RayBio Antibody Arrays (RayBiotech, Inc., Norcross, Ga.), CellCard (Vitra Bioscience, Mountain View, Calif.) and Quantum Dots' Mosaic (Invitrogen, Carlsbad, Calif.).

Particular embodiments can include control formulations (positive and/or negative), and/or one or more detectable labels. In particular embodiments, detectable labels that can be used for protein detection include radioactive isotopes or radiolabels (e.g., 32P and 13C), enzymes (e.g., luciferase, HRP and AP), dyes (e.g., rhodamine and cyanine), fluorescent tags or dyes (e.g., GFP, YFP, FITC), magnetic beads, or biotin. In particular embodiments, the detectable label is fluorescein, GFP, rhodamine, cyanine dyes, Alexa dyes, luciferase, and radiolabels, among others. Instructions for carrying out the assay, including, optionally, instructions for generating a score, can be included in the kit; e.g., written, tape, VCR, or CD-ROM.

In particular embodiments, the kits include materials and reagents necessary to conduct a methylation detection assay. In particular embodiments, the kits include materials and reagents necessary to conduct hybridization assays (e.g., PCR). In particular embodiments, the kits include materials and reagents necessary to conduct an immunoassay (e.g., ELISA). In particular embodiments, materials and reagents expressly exclude equipment (e.g., plate readers). In particular embodiments, kits can exclude materials and reagents commonly found in laboratory settings (pipettes; test tubes; distilled H2O).

The assayed sample can be any appropriate biological sample. In particular embodiments the sample is obtained from a subject diagnosed with PCa, and the sample includes the PCa. Cells and DNA from any biological sample(s) containing DNA can be used as a sample. Samples used for testing can be obtained from living or dead tissue and also archeological or forensic specimens containing cells or tissues. Exemplary samples include primary PCa tumor samples.

Particular embodiments disclosed herein include obtaining a sample from a subject having PCa; performing a methylation detection assay on the sample; determining one or more values based on the assaying; distinguishing metastatic-lethal PCa from indolent PCa based on the differential methylation status of a marker, as described elsewhere herein.

Particular embodiments also include predicting or diagnosing metastatic-lethal or indolent PCa in a subject by obtaining a sample from a subject suspected of having PCa; assaying the sample for methylation status of one or more markers disclosed herein; determining one or more marker values based on the assaying; comparing the one or more marker values to a reference level; and predicting or diagnosing metastatic-lethal or indolent PCa in the subject according to the methylation status of a marker as determined by the up- or down-regulation of the one or more markers, as described elsewhere herein.

A prediction or diagnosis according to the methods and kits disclosed herein can direct a treatment regimen. For example, a biological classification, prediction or diagnosis of metastatic-lethal PCa can direct a more aggressive or experimental treatment course. A classification, prediction or diagnosis of indolent PCa can direct a less aggressive or no further treatment course. Those of ordinary skill in the art classify treatments at a particular time as aggressive, experimental, moderate, minimal or “no” treatment based on a subject's prognosis and relevant standards and treatments at the time. For example, a treatment undergoing a clinical trial is an experimental treatment. Once the treatment is approved by a relevant regulatory agency within a jurisdiction, the treatment is no longer experimental in that jurisdiction.

In particular embodiments, the PCa epigenetic markers include one or more CpG methylation sites located in the gene FHAD1 (forkhead-associated (FHA) phosphopeptide binding domain 1). FHAD1 is a protein coding gene located on chromosome 1 of the human genome and is listed under Gene ID: 114827 in NCBI (see, e.g., SEQ ID NO: 1). In particular embodiments, differential methylation at CpG site cg02394978 in the FHAD1 gene is measured as an epigenetic marker of PCa (i.e, methylation levels at this CpG are lower in metastatic-lethal compared to indolent PCa).

In particular embodiments, the PCa epigenetic markers include one or more CpG methylation sites in the gene ALKBH5 (ALKB homolog 5). ALKBH5 is a gene that encodes a nucleic acid demethylase located on chromosome 17 and is listed under NCBI Gene ID: 54890 (see, e.g., SEQ ID NO: 2). In particular embodiments, methylation at CpG site cg07166550 in the ALKBH5 gene is measured as an epigenetic marker of PCa (i.e, methylation levels at this CpG are lower in metastatic-lethal compared to indolent PCa).

In particular embodiments, the PCa epigenetic markers include one or more CpG methylation sites in the gene KLHL8 (Kelch like family member 8), which encodes an adaptor protein involved in ubiquitination and is located on chromosome 4. KLHL8 is listed under NCBI Gene ID: 57563 (see, e.g., SEQ ID NO: 3). In particular embodiments, methylation at CpG site cg16713292 in the KLHL8 gene is measured as an epigenetic marker of PCa (i.e, methylation levels at this CpG are lower in metastatic-lethal compared to indolent PCa).

In particular embodiments, the PCa epigenetic markers include one or more CpG methylation sites in the gene ATP11A (ATPase, Class VI, type 11A), which is located on chromosome 13 and is listed under NCBI Gene ID: 23250 (see, e.g., SEQ ID NO: 4). In particular embodiments, methylation at CpG site cg21513610 in the ATP11A gene is measured as an epigenetic marker of PCa (i.e, methylation levels at this CpG are lower in metastatic-lethal compared to indolent PCa).

In particular embodiments, the PCa epigenetic markers include one or more CpG methylation sites in the gene PI15 (Peptidase Inhibitor), which is a gene encoding a membrane-bound ATPase located on chromosome 13. PI15 is listed under NCBI Gene ID: 51050 (see, e.g., SEQ ID NO: 5). In particular embodiments, methylation at CpG site cg24349665 in the PI15 gene is measured as an epigenetic marker of PCa (i.e, methylation levels at this CpG are higher in metastatic-lethal compared to indolent PCa).

In particular embodiments, the PCa epigenetic markers include one or more CpG methylation sites in Intergenic region 1. Intergenic region 1 is located on chromosome 17, in a region known as an open sea, meaning it is greater than 4 kb away from the nearest CpG island. In particular embodiments, methylation at CpG site cg01135464 in Intergenic region 1 is measured as an epigenetic marker of PCa (i.e, methylation levels at this CpG are higher in metastatic-lethal compared to indolent PCa).

In particular embodiments, the PCa epigenetic markers include one or more CpG methylation sites in Intergenic region 2. In particular embodiments, methylation at CpG site cg02223001 in Intergenic region 2 is measured as an epigenetic marker of PCa (i.e, methylation levels at this CpG are higher in metastatic-lethal compared to indolent PCa).

In particular embodiments, the PCa epigenetic markers include one or more CpG methylation sites in Intergenic region 3. Intergenic region 3 is located on chromosome 1, in a region known as a shore, meaning it is adjacent to or less than 2 kb away from the nearest CpG island. In particular embodiments, methylation at CpG site cg22501793 in Intergenic region 3 is measured as an epigenetic marker of PCa (i.e, methylation levels at this CpG are higher in metastatic-lethal compared to indolent PCa).

The disclosure is not limited to the particularly referenced gene and associated nucleic acid and protein sequences but instead also encompasses sequences including 80% sequence identity; 81% sequence identity; 82% sequence identity; 83% sequence identity; 84% sequence identity; 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity or 99% sequence identity to a gene sequence referenced herein.

“% sequence identity” refers to a relationship between two or more sequences, as determined by comparing the sequences. In the art, “identity” also means the degree of sequence relatedness between sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity can be found in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. “Default values” mean any set of values or parameters which originally load with the software when first initialized.

Moreover, the term “gene” can include not only coding sequences but also regulatory regions such as promoters, enhancers, and termination regions. The term further can include all introns and other DNA sequences spliced from the mRNA transcript, along with variants resulting from alternative splice sites. Portions of complete gene sequences can be referenced as is understood by one of ordinary skill in the art.

The Exemplary Embodiments and Examples below are included to demonstrate particular embodiments of the disclosure. Those of ordinary skill in the art should recognize in light of the present disclosure that many changes can be made to the particular embodiments disclosed herein and still obtain a like or similar result without departing from the spirit and scope of the disclosure.

### Exemplary Embodiments

1. A method to distinguish metastatic-lethal prostate cancer (PCa) from indolent PCa including:

obtaining a tumor tissue sample;

detecting methylation status of cytosines located in one or more epigenetic loci selected from Intergenic 1, Intergenic 2, FHAD1, ALKBH5, KLHL8, ATP11A, Intergenic 3, and PI15;

and

distinguishing metastatic-lethal PCa from indolent PCa based on the detecting.

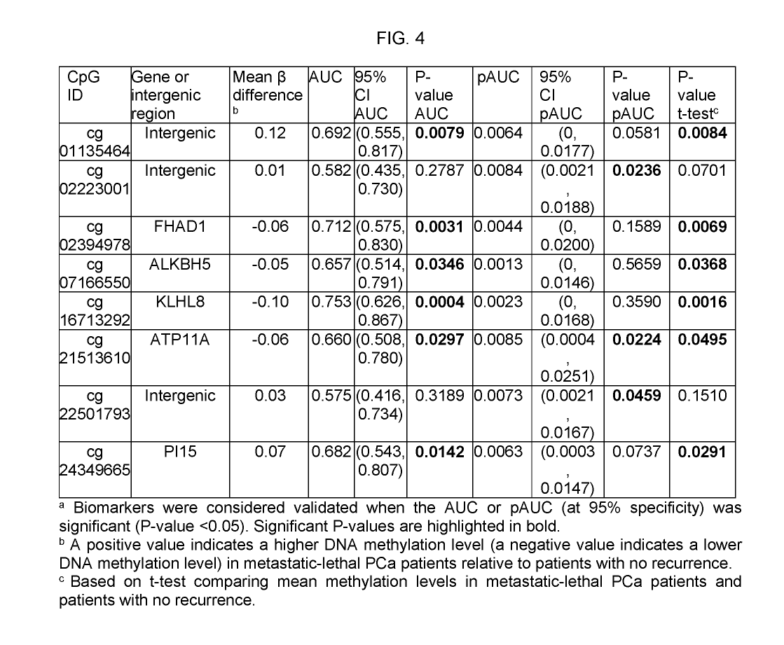

2. A method of embodiment 1 wherein the cytosines are part of CpG pairs.
3. A method of embodiment 2 wherein the CpG pairs are within CpG islands.
4. A method of embodiment 1 or 2 wherein the cytosines are within CpG site: cg01135464; CpG site: cg02223001; CpG site: cg02394978; CpG site: cg07166550; CpG site: cg16713292; CpG site: cg21513610; CpG site: cg22501793; and/or CpG site: cg24349665,
5. A method of embodiment 1 or 2 wherein the cytosines are all cytosines within CpG pairs within the selected gene or intergenic region.
6. A method of any of embodiments 1-5 wherein a reference level is derived from a population of subjects with indolent PCa.
7. A method of embodiment 6 wherein up-regulation of methylation status at Intergenic 1, Intergenic 2, Intergenic 3, and/or PI15 as compared to the reference level distinguishes the sample as metastatic-lethal PCa.
8. A method of embodiment 6 wherein lack of a statistically-significant difference in methylation status at Intergenic 1, Intergenic 2, Intergenic 3, and/or PI15 as compared to the reference level distinguishes the sample as indolent PCa.
9. A method of embodiment 7 or 8 wherein down-regulation of methylation status at FHAD1, ALKBH5, KLHL8, and/or ATP11A as compared to the reference level distinguishes the sample as metastatic-lethal PCa.
10. A method of embodiment 6 or 8 wherein lack of a statistically-significant difference in methylation status at FHAD1, ALKBH5, KLHL8, and/or ATP11A as compared to the reference level distinguishes the sample as indolent PCa.
11. A method of any of embodiments 1-5 wherein the reference level is derived from a population of subjects with metastatic-lethal PCa.
12. A method of embodiment 11 wherein down-regulation of methylation status at Intergenic 1, Intergenic 2, Intergenic 3, and/or PI15 as compared to the reference level distinguishes the sample as indolent PCa.
13. A method of embodiment 11 wherein lack of a statistically-significant difference in methylation status at Intergenic 1, Intergenic 2, Intergenic 3, and/or PI15 as compared to the reference level distinguishes the sample as metastatic-lethal PCa.
14. A method of embodiment 11 or 12 wherein up-regulation of methylation status at FHAD1, ALKBH5, KLHL8, and/or ATP11A as compared to the reference level distinguishes the sample as indolent PCa.
15. A method of embodiment 11 or 13 wherein lack of a statistically-significant difference in methylation status at FHAD1, ALKBH5, KLHL8, and/or ATP11A as compared to the reference level distinguishes the sample as metastatic-lethal PCa.
16. A method of any of embodiments 1-15 wherein the method includes detecting methylation status of cytosines located in Intergenic 1; Intergenic 2; FHAD1; ALKBH5; KLHL8; ATP11A; Intergenic 3; and PI15.
17. A method of any of embodiments 1-15 wherein the method includes detecting methylation status of cytosines located in Intergenic 1 and the methylation status of cytosines located in one or more of Intergenic 2; FHAD1; ALKBH5; KLHL8; ATP11A; Intergenic 3; and PI15.
18. A method of any of embodiments 1-15 wherein the method includes detecting methylation status of cytosines located in Intergenic 2 and the methylation status of cytosines located in one or more of Intergenic 1; FHAD1; ALKBH5; KLHL8; ATP11A; Intergenic 3; and PI15.
19. A method of any of embodiments 1-15 wherein the method includes detecting methylation status of cytosines located in FHAD1 and the methylation status of cytosines located in one or more of Intergenic 1; Intergenic 2; ALKBH5; KLHL8; ATP11A; Intergenic 3; and PI15.
20. A method of any of embodiments 1-15 wherein the method includes detecting methylation status of cytosines located in ALKBH5 and the methylation status of cytosines located in one or more of Intergenic 1; Intergenic 2; FHAD1; KLHL8; ATP11A; Intergenic 3; and PI15.
21. A method of any of embodiments 1-15 wherein the method includes detecting methylation status of cytosines located in KLHL8 and the methylation status of cytosines located in one or more of Intergenic 1; Intergenic 2; FHAD1; ALKBH5; ATP11A; Intergenic 3; and PI15.
22. A method of any of embodiments 1-15 wherein the method includes detecting methylation status of cytosines located in ATP11A and the methylation status of cytosines located in one or more of Intergenic 1; Intergenic 2; FHAD1; ALKBH5; KLHL8; Intergenic 3; and PI15.
23. A method of any of embodiments 1-15 wherein the method includes detecting methylation status of cytosines located in Intergenic 3 and the methylation status of cytosines located in one or more of Intergenic 1; Intergenic 2; FHAD1; ALKBH5; KLHL8; ATP11A; and PI15.
24. A method of any of embodiments 1-15 wherein the method includes detecting methylation status of cytosines located in PI15 and the methylation status of cytosines located in one or more of Intergenic 1; Intergenic 2; FHAD1; ALKBH5; KLHL8; ATP11A; and Intergenic 3.
25. A method of any of embodiments 1-15 wherein the method includes detecting methylation status of cytosines located in one or more of ALKBH5, FHAD1, KLHL8 and PI15 and wherein the methylation status improves the prognostic discrimination of a Gleason score (e.g., by improving ROC score).
26. A method of embodiment 25 wherein the Gleason score is <=6, a 3+4=7 or a 4+3=7, or 8-10 Gleason score.
27. A method of any of embodiments 1-26 wherein the sample is obtained from a subject.
28. A method of any of embodiments 1-27 wherein the sample is a primary PCa tumor sample.
29. A method of any of embodiments 1-28 wherein methylation status is defined by a mean β difference (e.g., see FIG. 4) or a β difference.
30. A method of embodiment 25 wherein the mean β difference or β difference is (+) or (−).
31. A method of any of embodiments 1-5, 7, 9, 11, 13, 15, or 16-30 wherein biological classification as metastatic-lethal PCa directs an aggressive or experimental treatment.
32. A method of any of embodiments 1-6, 8, 10, 12, 14, or 16-30 wherein biological classification as indolent PCa directs moderate, minimal, or no treatment.
33. A method of any of embodiments 1-32 wherein the detecting includes assaying using a bisulfite based methylation assay.
34. A method of any of embodiments 1-33 wherein the detecting includes assaying using pyrosequencing.
35. A kit for distinguishing metastatic-lethal PCa from indolent PCa including reagents to detect methylation status of cytosines located in one or more epigenetic loci selected from Intergenic 1, Intergenic 2, FHAD1, ALKBH5, KLHL8, ATP11A, Intergenic 3, and PI15.
36. A kit of embodiment 35 wherein the reagents detect methylation status of cytosines that are part of CpG pairs.
37. A kit of embodiment 36 wherein the reagents detect methylation status of cytosines that are part of CpG pairs within CpG islands.
38. A kit of embodiment 35 or 36 wherein the reagents detect methylation status of cytosines within CpG site: cg01135464; CpG site: cg02223001; CpG site: cg02394978; CpG site: cg07166550; CpG site: cg16713292; CpG site: cg21513610; CpG site: cg22501793; and/or CpG site: cg24349665.
39. A kit of embodiment 35 or 36 wherein the reagents detect methylation status of cytosines within all CpG pairs within the selected gene or intergenic region.
40. A kit of any of embodiments 35-39 including a reference level.
41. A kit of embodiment 40 wherein the reference level is derived from a population of subjects with indolent PCa.
42. A kit of embodiment 41 wherein up-regulation of methylation status at Intergenic 1, Intergenic 2, Intergenic 3, and/or PI15 as compared to the reference level distinguishes the sample as metastatic-lethal PCa.
43. A kit of embodiment 41 wherein lack of a statistically-significant difference in methylation status at Intergenic 1, Intergenic 2, Intergenic 3, and/or PI15 as compared to the reference level distinguishes the sample as indolent PCa.
44. A kit of embodiment 41 or 42 wherein down-regulation of methylation status at FHAD1, ALKBH5, KLHL8, and/or ATP11A as compared to the reference level distinguishes the sample as metastatic-lethal PCa.
45. A kit of embodiment 41 or 43 wherein lack of a statistically-significant difference in methylation status at FHAD1, ALKBH5, KLHL8, and/or ATP11A as compared to the reference level distinguishes the sample as indolent PCa.
46. A kit of embodiment 40 wherein the reference level is derived from a population of subjects with metastatic-lethal PCa.
47. A kit of embodiment 46 wherein down-regulation of methylation status at Intergenic 1, Intergenic 2, Intergenic 3, and/or PI15 as compared to the reference level distinguishes the sample as indolent PCa.
48. A kit of embodiment 46 wherein lack of a statistically-significant difference in methylation status at Intergenic 1, Intergenic 2, Intergenic 3, and/or PI15 as compared to the reference level distinguishes the sample as metastatic-lethal PCa.
49. A kit of embodiment 46 or 47 wherein up-regulation of methylation status at FHAD1, ALKBH5, KLHL8, and/or ATP11A as compared to the reference level distinguishes the sample as indolent PCa.
50. A kit of embodiment 46 or 49 wherein lack of a statistically-significant difference in methylation status at FHAD1, ALKBH5, KLHL8, and/or ATP11A as compared to the reference level distinguishes the sample as metastatic-lethal PCa.
51. A kit of any of embodiments 35-50 wherein the kit includes reagents to detect methylation status of cytosines located in Intergenic 1; Intergenic 2; FHAD1; ALKBH5; KLHL8; ATP11A; Intergenic 3; and PI15.
52. A kit of any of embodiments 35-50 wherein the kit includes reagents to detect methylation status of cytosines located in Intergenic 1 and methylation status of cytosines located in one or more of Intergenic 2; FHAD1; ALKBH5; KLHL8; ATP11A; Intergenic 3; and PI15.
53. A kit of any of embodiments 35-50 wherein the kit includes reagents to detect methylation status of cytosines located in Intergenic 2 and methylation status of cytosines located in one or more of Intergenic 1; FHAD1; ALKBH5; KLHL8; ATP11A; Intergenic 3; and PI15.
54. A kit of any of embodiments 35-50 wherein the kit includes reagents to detect methylation status of cytosines located in FHAD1 and methylation status of cytosines located in one or more of Intergenic 1; Intergenic 2; ALKBH5; KLHL8; ATP11A; Intergenic 3; and PI15.
55. A kit of any of embodiments 35-50 wherein the kit includes reagents to detect methylation status of cytosines located in ALKBH5 and methylation status of cytosines located in one or more of Intergenic 1; Intergenic 2; FHAD1; KLHL8; ATP11A; Intergenic 3; and PI15.
56. A kit of any of embodiments 35-50 wherein the kit includes reagents to detect methylation status of cytosines located in KLHL8 and methylation status of cytosines located in one or more of Intergenic 1; Intergenic 2; FHAD1; ALKBH5; ATP11A; Intergenic 3; and PI15.
57. A kit of any of embodiments 35-50 wherein the kit includes reagents to detect methylation status of cytosines located in ATP11A and methylation status of cytosines located in one or more of Intergenic 1; Intergenic 2; FHAD1; ALKBH5; KLHL8; Intergenic 3; and PI15.
58. A kit of any of embodiments 35-50 wherein the kit includes reagents to detect methylation status of cytosines located in Intergenic 3 and methylation status of cytosines located in one or more of Intergenic 1; Intergenic 2; FHAD1; ALKBH5; KLHL8; ATP11A; and PI15.
59. A kit of any of embodiments 35-50 wherein the kit includes reagents to detect methylation status of cytosines located in PI15 and methylation status of cytosines located in one or more of Intergenic 1; Intergenic 2; FHAD1; ALKBH5; KLHL8; ATP11A; and Intergenic 3.
60. A kit of any of embodiments 35-59 including reagents to perform a bisulfite-based methylation assay.
61. A kit of embodiment 35-60 further including reagents to perform pyrosequencing.
62. A kit of any of embodiments 35-61 including probes and silicon based arrays to perform a methylation assay.
63. A kit of embodiment 62 wherein the probes include beads.
64. A kit of embodiment 63 wherein the beads allow a detectable label to be bound.
65. A kit of any of embodiments 35-64 including proteins and/or nucleotide sequences that bind to one or more proteins encoded by, and/or one or more nucleotide sequences corresponding to, one or more of Intergenic 1; Intergenic 2; FHAD1; ALKBH5; KLHL8; ATP11A; Intergenic 3; and PI15.
66. A kit of any of embodiments 35-65 including a DNA nucleotide sequence and/or a RNA nucleotide sequence.
67. A kit of embodiment 66 wherein the proteins include antibodies, epitopes, or mimitopes.
68. A kit of any of embodiments 35-67 including a detectable label.
69. A kit of embodiment 68 wherein the detectable label is a radioactive isotope, enzyme, dye, fluorescent dye, magnetic bead, or biotin.
70. A kit of any of embodiments 35-69 including bisulfate and PCR primers specific for bisulfite-converted DNA.
71. A kit of any of embodiments 35-70 including DNA-fragmenting enzymes.
72. A kit of any of embodiments 35-71 including hybridization buffer.
73. A kit of any of embodiments 35-72 including target-specific probes for CpG pairs within any of SEQ ID NOs: 1-5.
74. A kit of any of embodiments 35-73 including beads with target-specific probes for CpG pairs within CpG site: cg01135464; CpG site: cg02223001; CpG site: cg02394978; CpG site: cg07166550; CpG site: cg16713292; CpG site: cg21513610; CpG site: cg22501793; and/or CpG site: cg24349665.
75. A kit of any of embodiments 35-74 including one or more nucleotides including one or more of SEQ ID NOs: 6-29.
76. A method or kit to improve the prognostic determination of a Gleason score including practicing a method or using a kit of any of embodiments 1-75.

Examples. Methods. Study Subjects. Fred Hutchinson (FH) Cancer Research Center Cohort. The FH cohort includes 510 European-American PCa patients who underwent radical prostatectomy as primary therapy for clinically localized adenocarcinoma of the prostate. These patients were previously enrolled in population-based studies. Agalliu et al., Am J Epidemiol 168:250-60, 2008; Stanford et al., Cancer Epidemiol Biomarkers Prev 8:881-6, 1999. The first study included men ages 40-64 years who were diagnosed between January 1993 and December 1996, and in the second study, men were ages 35-74 years and were diagnosed between January 2002 and December 2005. Gleason score, diagnostic PSA, and tumor stage were collected from the Seattle-Puget Sound Surveillance, Epidemiology, and End Results Program cancer registry. Vital status and underlying cause of death were also obtained from the cancer registry, and cause of death was confirmed by review of death certificates. PCa recurrence status was determined from prospectively collected information from follow-up surveys that were completed by patients in 2004-2005 and in 2010-2011, review of medical records, and/or physician follow-up as needed. Metastatic progression was confirmed by positive bone scan, MRI, CT or biopsy. PCa-specific deaths included those with underlying cause of death attributed to ICD-9 code 180.0 or ICD-10 code C61.9. Patients who developed metastases or died from PCa were combined in a lethal phenotype category. Over a mean follow-up of 8.2 years, 317 patients had no evidence of recurrence and 113 had recurred, including 27 metastatic-lethal events; recurrence status was unknown for 80 men. For the present analysis, the lethal phenotype patients were compared to patients who had not recurred. The FH Institutional Review Board approved the study and all participants signed informed consent statements.

Eastern Virginia (EV) Medical School Cohort. The validation cohort included 80 patients diagnosed with localized stage PCa who underwent radical prostatectomy at EV Medical School. The cohort included men who experienced disease progression to metastatic or lethal PCa (n=31) and patients with no evidence of recurrence for five or more years after diagnosis (n=49) who were matched based on diagnosis years (1992-2009). Metastatic-lethal events were identified as described in the FH cohort, and all patients were European-Americans. The patients in the EV cohort were followed for outcomes on average for 9.0 years.

Tumor Tissue Sample Preparation and DNA Extraction. Formalin-fixed paraffin-embedded prostate tumor tissue blocks were obtained from radical prostatectomy specimens for both cohorts and used to make hematoxalin and eosin stained slides, which were reviewed by pathologists to confirm the presence and location of adenocarcinoma. For each patient two 1-mm tumor tissue cores from the dominant lesion that were enriched with 75% tumor cells were taken for DNA purification. The RecoverAll Total Nucleic Acid Isolation Kit (Ambion/Applied Biosciences, Austin, Tex.) was used to extract DNA, which was then quantified with PicoGreen, aliquoted onto 96-well plates and shipped to Illumina (Illumina, Inc., San Diego, Calif.) for DNA methylation profiling.

DNA Methylation Profiling. The EZ DNA Methylation Kit (Zymo Research, Irvine, Calif.) was used to bisulfite convert tumor DNA samples. Controls on the array were used to track the bisulfite conversion efficiency. The Infinium® HumanMethylation450 BeadChip (Illumina) was used to measure epigenome-wide methylation using beads with target-specific probes designed to interrogate individual CpG sites (>485,000). Bibikova et al., Genomics 98:288-95, 2011. Samples from the FH cohort were assayed as one batch (7 plates) and the EV samples were assayed as a second batch (2 plates). Across the 96-well plates, blind duplicate (FH, n=16; EV, n=7) and replicate (FH, n=2; EV, n=3) samples were incorporated for each cohort. All plates also contained Illumina controls and two negative controls. PCa outcome events were randomly distributed across plates, and laboratory personnel were blinded to the location of duplicate and replicate samples.

Data Processing. Failed samples were identified by using the detection P-value metric according to Illumina protocols. A sample was excluded if less than 95% of the CpG sites for that sample on the array were detected with a detection P-value <0.05, resulting in removal of 31 FH and 15 EV samples. The final number of patients in the FH cohort and EV cohort was 327 (303 non-recurrent, 24 metastatic-lethal) and 65 (41 non-recurrent, 24 metastatic-lethal), respectively. Further, CpG sites with a detection P-value of >0.01 were excluded. After data filtering, 478,998 CpGs were available in the FH cohort and 479,103 in the EV cohort (477,460 overlapped). Correlation coefficients for duplicate samples in the FH and EV cohorts were 0.96-0.99 and 0.99, respectively. The correlation coefficients for replicate samples in FH and EV were 0.99 and 0.98.

Gene Expression Profiling. The same patients' tumor samples were used for mRNA expression profiling using the Whole-Genome DASL® HT Assay (Illumina). Transcript correlations between duplicated samples (19 pairs) ranged from 0.96 to 0.99. In addition, replicate tumor RNA samples (6 pairs) were included, and the transcript correlations across plates were 0.95-0.99. There were 353 patients with DNA methylation and mRNA expression data.

Statistical Analysis. Batch effects were removed using Combat (Johnson et al., Biostatistics 8:118-27, 2007), and the methylation data were normalized using subset-quantile within array normalization (Bioconductor minfi). Maksimovic et al., Genome Biol 13:R44, 2012. Methylation β- and M-values were calculated where β-values represent the percentage of DNA methylation at a CpG site. Methylation M-values are the logit transformed β-values that are normally distributed. M-values were used for statistical testing and β-values to represent methylation differences between patient groups. Genome annotation of the CpGs was based on the Illumina protocol. Hansen, IlluminaHumanMethylation450kanno.ilmn12.hg19: Annotation for Illumina's 450 k methylation arrays. R package version 0.2.1. DNA methylation biomarkers for prognosis were identified using the FH cohort. First, for all individual CpG sites, the AUC and partial AUC (pAUC) for predicating metastatic-lethal versus non-recurrent PCa were calculated. The pAUC evaluates performance at fixed high (95%) specificity as selection of biomarkers with a low false-positive rate was the aim. The top 4% of markers based on pAUC and the top 1% based on AUC were selected, yielding 22,290 CpGs for further analysis.

Next, the biomarkers that showed the greatest improvement in predicting metastatic-lethal PCa compared to Gleason score alone were identified. Because Gleason score is the most widely used measure of tumor aggressiveness, identification of CpGs that could improve the prognostic discrimination of patients beyond that provided by Gleason score was the aim. Other potential prognostic classifiers were also considered including age at diagnosis, diagnostic PSA, and pathologic stage, but these factors did not improve the prediction of metastatic-lethal PCa compared to models with Gleason score only (P>0.05), and were therefore not considered in further analyses.

A logistic regression model for metastatic-lethal versus non-recurrent PCa was fit containing Gleason score as the only predictor. Based on that model, forward model selection was done using three selection criteria: AUC, pAUC (95% specificity), and P-value (Wald test). For each criterion, the CpG that showed the greatest improvement or was the most significant in predicting metastatic-lethal PCa compared to the base model with Gleason score alone was identified; the identified biomarker was then added to the model with Gleason score. Forward selection was continued, each time selecting one additional CpG to be included in the model, until a pre-specified stopping criterion was met; for AUC this was an increment of AUC <0.005; for pAUC this was an increment of pAUC <0.0005; and for P-value this was >0.05. This entire process was repeated 100 times with bootstrap samples. The biomarkers that were selected multiple times in the different bootstrap cohorts (≥3 when considering AUC; when considering pAUC; ≥4 when considering P-value) were chosen for further evaluation.

The methylation biomarkers that were most predictive for metastatic-lethal disease in the FH cohort were then tested in the EV (validation) cohort. For each biomarker the AUC and pAUC (95% specificity) for metastatic-lethal versus non-recurrent PCa was calculated. P-values for AUC and pAUC were computed using 10,000 permutations, and 95% confidence intervals for AUC and pAUC were calculated using 2,000 stratified bootstrap replicates. Likelihood ratio tests were also computed to compare models fit with Gleason score and a CpG biomarker compared to a model with Gleason score only. All statistical analyses were conducted using R.

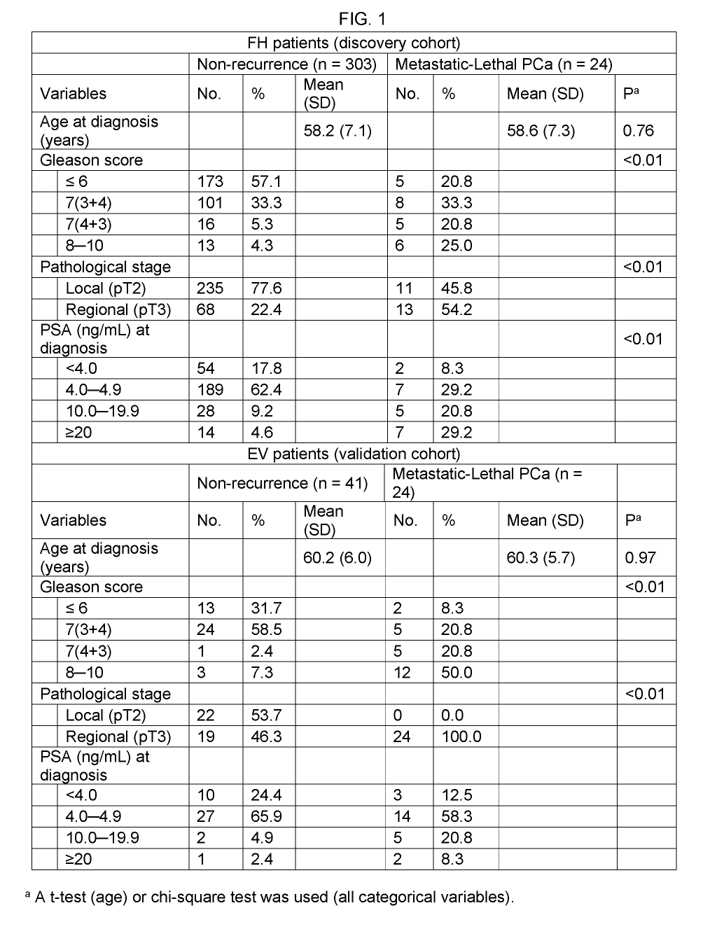

Results. There was no difference in mean age between patients with metastatic-lethal PCa compared to those who did not recur in either cohort (FIG. 1). In both cohorts, Gleason score, pathological stage, and PSA at diagnosis were higher in men with the lethal phenotype relative to men with no evidence of recurrence (all P-values <0.01).

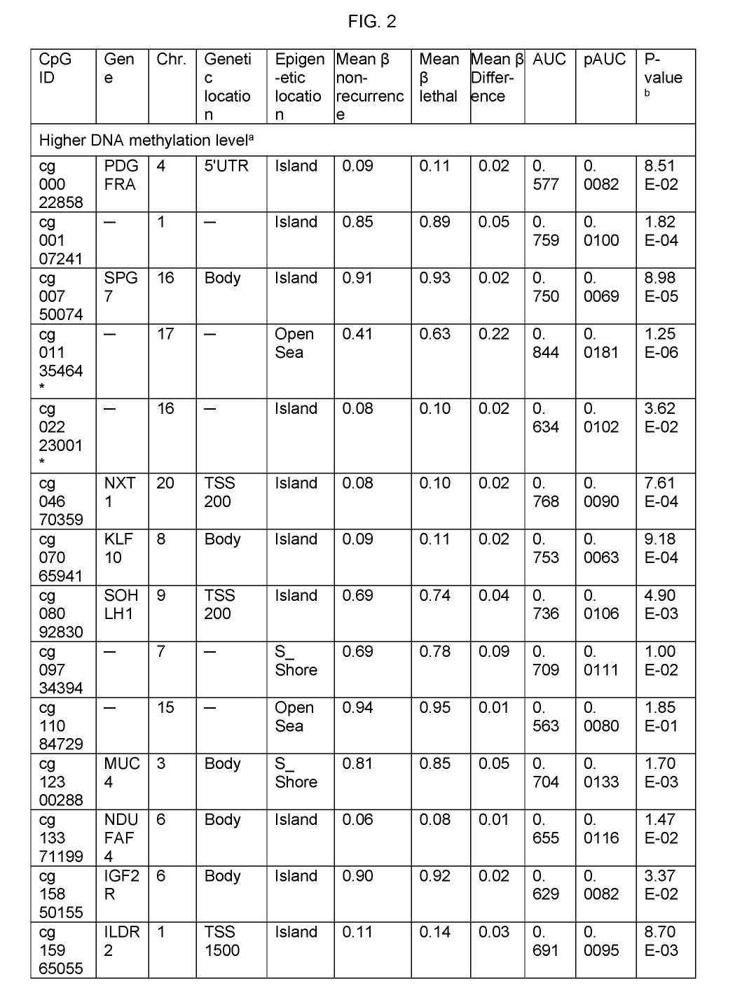

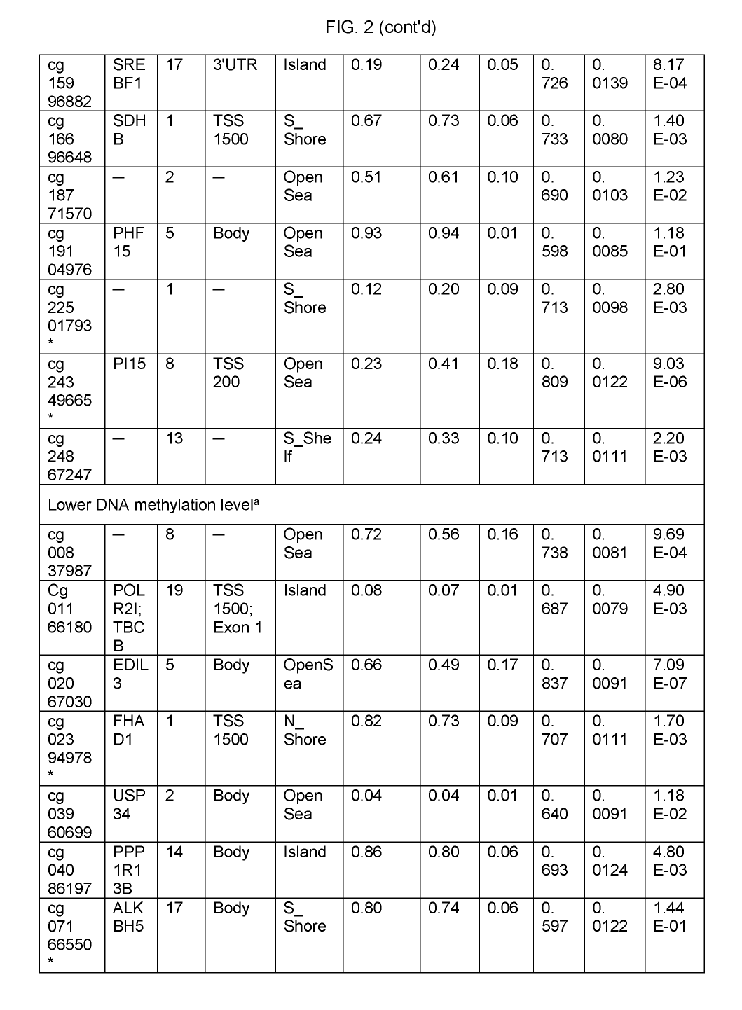

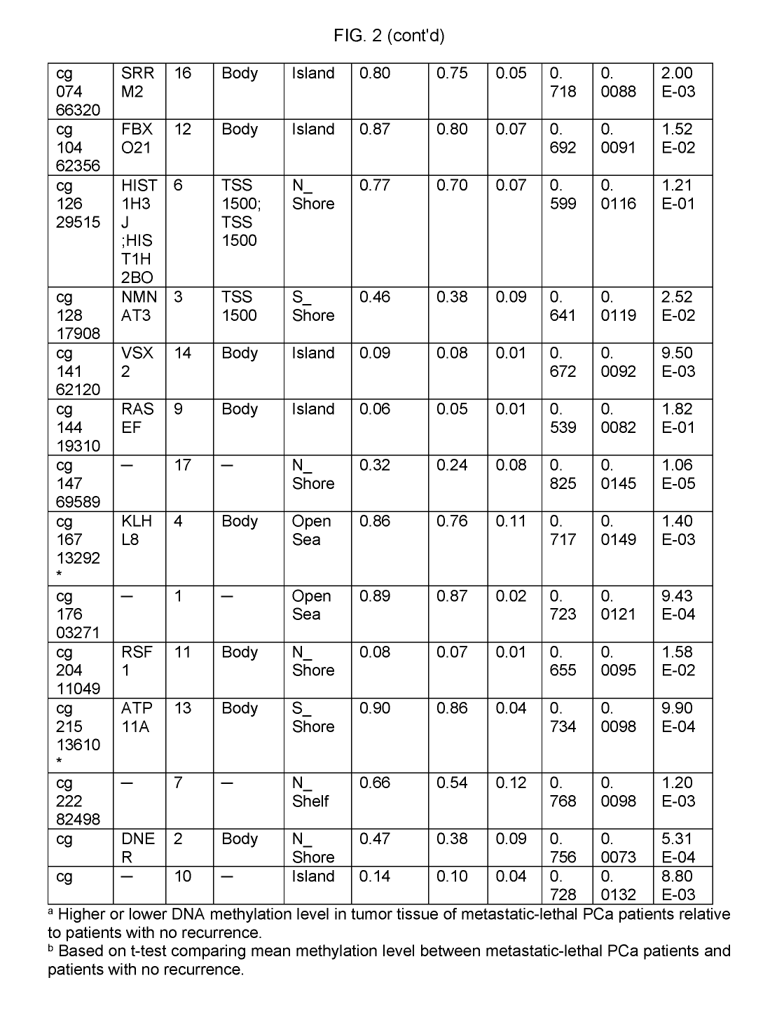

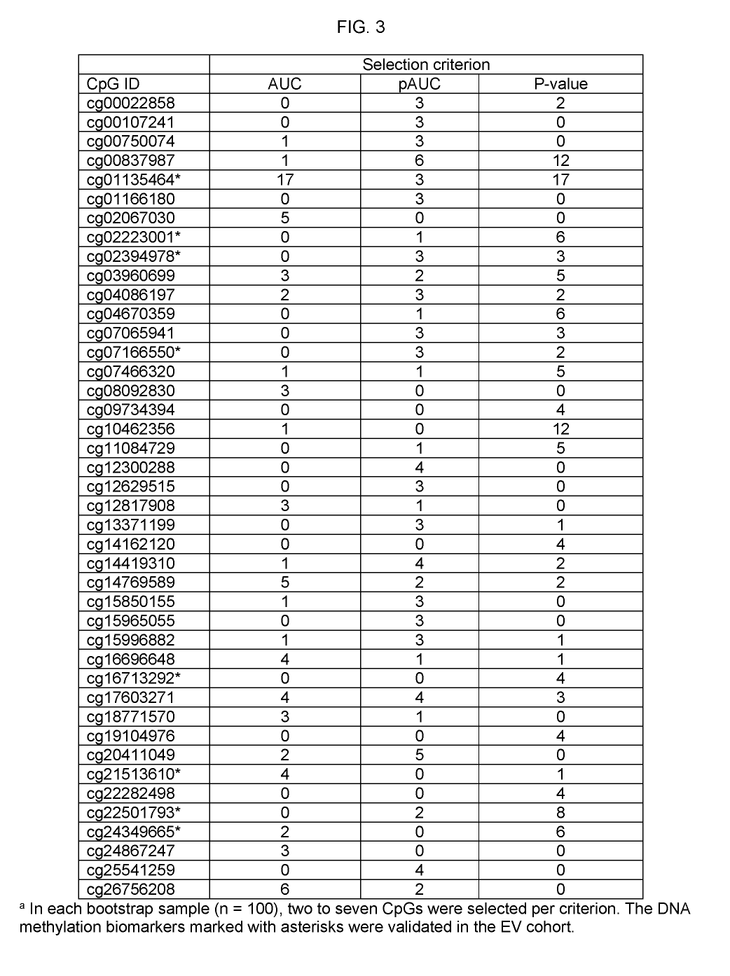

FIG. 2 shows the 42 DNA methylation biomarkers that were most predictive for metastatic-lethal PCa in the FH cohort. These CpGs were identified based on their ability to improve the prognostic discrimination beyond Gleason score alone (FIG. 3), and a subset of the CpG biomarkers were validated in the EV cohort (FIG. 2, asterisks). Half of the 42 biomarkers showed higher methylation in metastatic-lethal PCa compared to non-recurrent disease (FIG. 2). The 42 biomarkers had a mean methylation difference between patient groups (metastatic-lethal vs. non-recurrent) that ranged from 1% to 22% (average=6.1%), and pAUC and AUC values for metastatic-lethal PCa ranged from 0.0063 to 0.0181 and 0.539 to 0.844, respectively. DNA methylation levels of the 42 biomarkers were not strongly correlated (all pairwise r2<0.5).

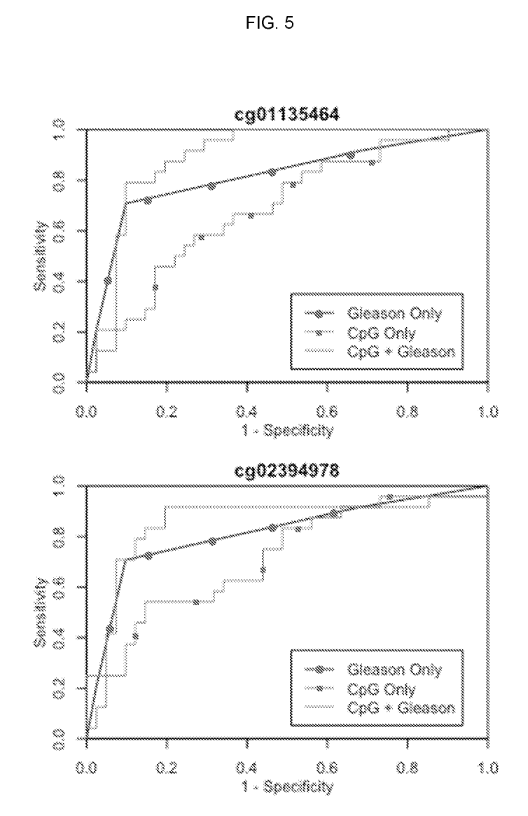

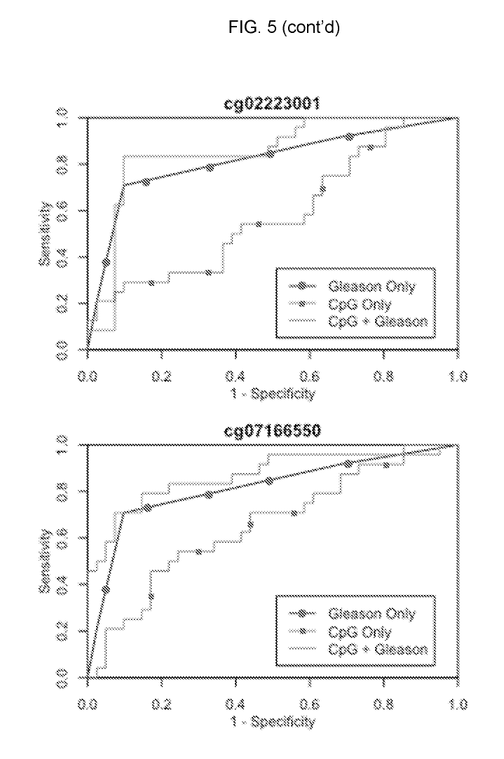

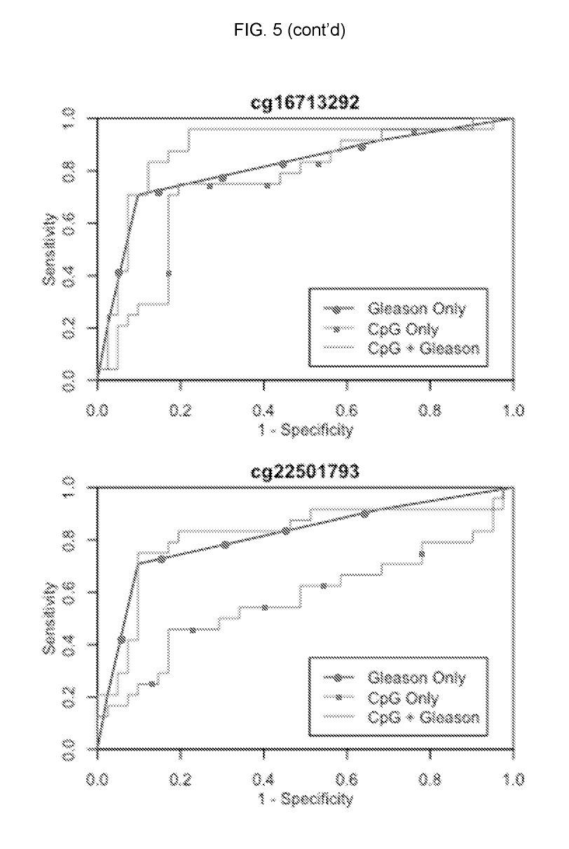

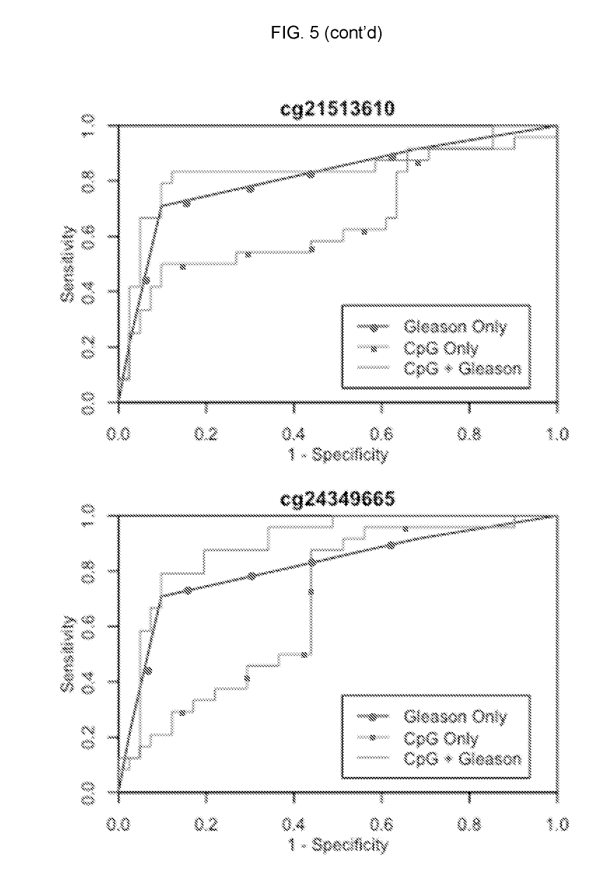

The 42 top-ranked biomarkers were next evaluated in the EV cohort. For 30 of the CpGs, the difference in methylation level between metastatic-lethal vs. non-recurrent PCa was in the same direction in the EV as in the FH cohort. Eight of these biomarkers demonstrated a significant AUC or pAUC in the EV cohort (P-value <0.05; FIG. 4). One of the biomarkers had both a significant AUC and pAUC (ATP11A cg21513610). The CpG with the largest mean methylation difference was cg01135464. The biomarker with the highest AUC was KLHL8 cg16713292 (0.753), and the largest pAUC was for ATP11A cg21513610 (0.0085). Whether methylation levels of these CpGs were correlated with methylation levels of adjacent CpGs in the same gene or intergenic region was next investigated. For five of the CpGs the methylation levels were correlated (pairwise r²>0.5) with methylation levels of nearby CpG sites (79 of 347 CpGs in ATP11A; 1 of 33 CpGs in FHAD1; 3 of 6 CpGs in PI15; 2 of 2 CpGs near cg01135464 [Chr. 17, OpenSea]; and 1 of 2 CpGs near cg22501793 [Chr. 1, S_Shore]). FIG. 5 shows the ROC curves for each of the eight biomarkers alone, and combined with Gleason score.

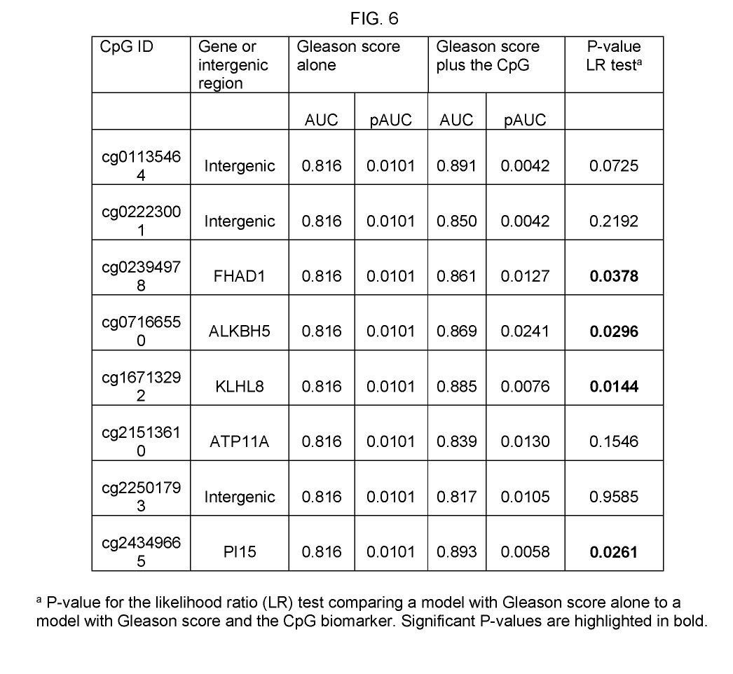

FIG. 6 shows the performance of the eight validated biomarkers evaluated for classifying metastatic-lethal PCa when combined with Gleason score. The AUC for Gleason score alone in the EV cohort was 0.816. This is higher than what has been reported in other studies and likely reflects the study design, which involved selecting metastatic-lethal patients and a group of patients without evidence of recurrence. Gleason score had a pAUC for metastatic-lethal PCa of 0.0101. Likelihood ratio tests were then performed comparing the model with Gleason score only to a model that included both Gleason score and one of the eight CpGs. This test was significant for four of the CpGs (P<0.05): ALKBH5 (cg07166550), FHAD1 (cg02394978), KLHL8 (cg16713292), and PI15 (cg24349665), providing further evidence that they are complementary to Gleason score for the prognostic discrimination of patients.

In a final analysis tumor mRNA expression profiles were evaluated. For two of the five genes that encompassed a validated biomarker, DNA methylation levels were significantly correlated with transcript levels: ATP11A (Pearson r²=−0.29, P=2.78E-18) and PI15 (Pearson r²=−0.28, P=5.77E-08). ATP11A cg21513610 is located in the gene body, whereas cg24349665 is in the promoter region of PI15.

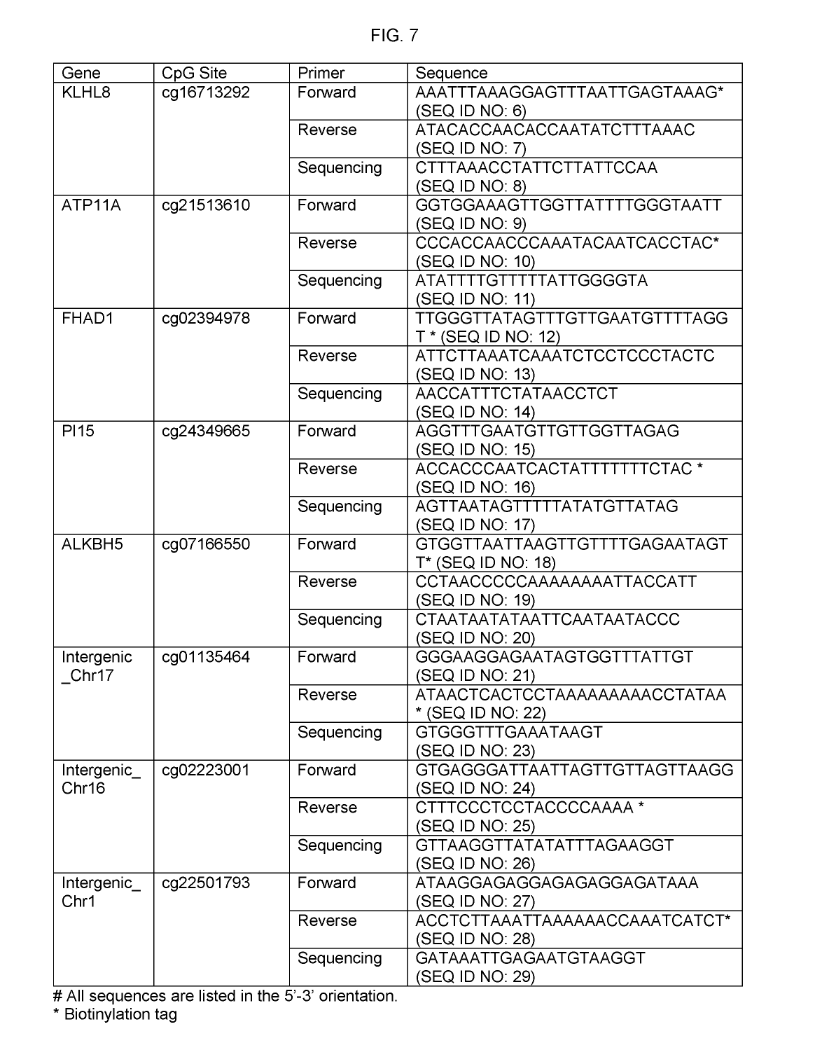

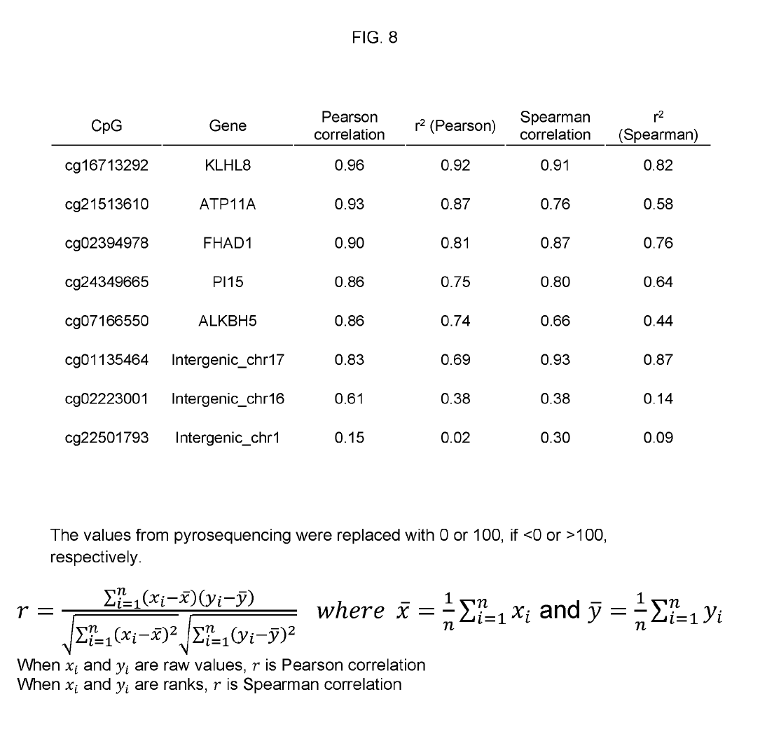

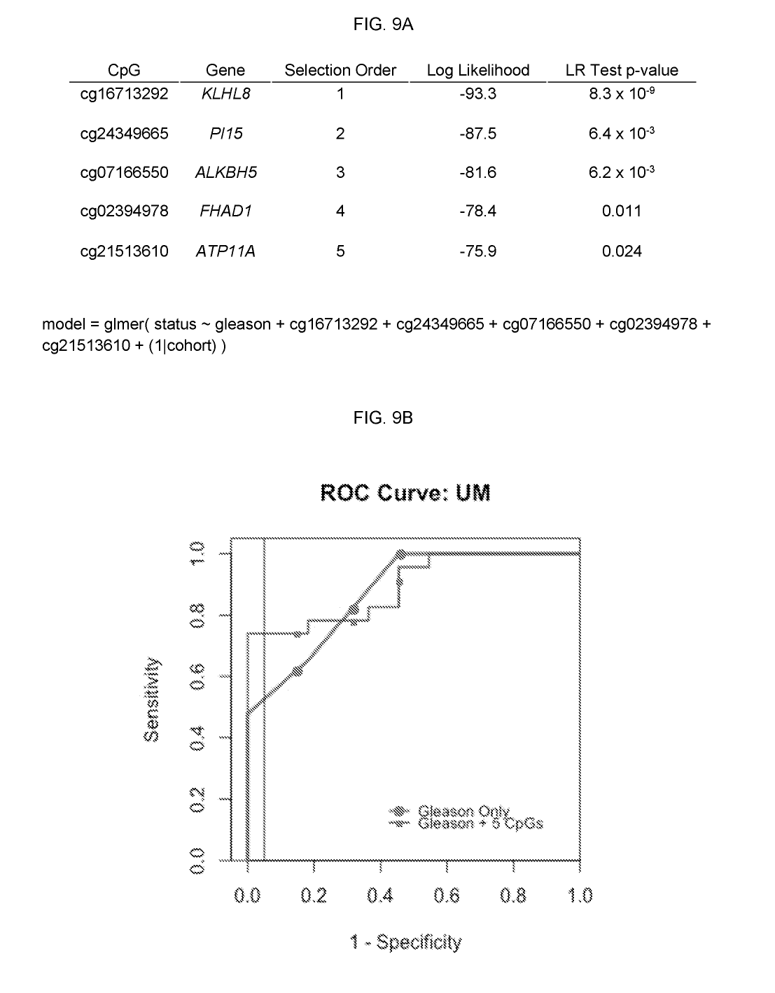

Technical validation of the markers was performed using pyrosequencing. Primers used for the pyrosequencing-based DNA methylation assay are shown in FIG. 7. First, methylation assay results were compared to the results obtained using the HM450 technique. The pyrosequencing results highly correlated with the HM450 results for the five CpG markers within genes (FIG. 8), indicating that pyrosequencing is a technique that can be used for the disclosed methods and kits for distinguishing metastatic-lethal and indolent PCa. Model training was conducted using the FH and EV cohorts and tested using samples from a University of Michigan cohort, and the selection order of each of the markers in the model was based on Log likelihood and LR Test p values for each marker, which are shown in FIG. 8. Next, forward model building with the Gleason score was performed for the five CpGs that were validated, and all five CpGs were selected into the model (FIG. 9A). The fitted model results are shown in FIG. 9B. The AUC for the model plus Gleason score was 0.89, which was higher than the AUC for Gleason score alone (0.87). These data demonstrate the utility of the markers, and indicate that pyrosequencing is a technique that can be utilized to assay for DNA methylation to help distinguish between indolent and metastatic-lethal PCa.

The methylation status of the epigenetic markers disclosed herein was detected and confirmed using two distinct assay types—the HM450 array and pyrosequencing.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in ability to detect metastatic-lethal or indolent PCa in a subject based on detecting methylation status of the CpG sites identified herein.

In the context of nucleotide sequences, “reagents to detect”, “target specific probes” and “specific for” mean that the nucleotide sequences interact with target sequences (or sequences related to the target sequence based on the particular assay) with sufficient specificity and strength to reliably detect methylation status of cytosines within the targeted sequence. Particular nucleotide sequences with these characteristics can be readily generated and identified by those of ordinary skill in the art, based on the teachings of the current disclosure and with reference to numerous publicly available resources and databases.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that can be employed are within the scope of the invention. Thus, by way of example, but not of limitation, alternative configurations of the present invention can be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention can be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method to distinguish metastatic-lethal prostate cancer (PCa) from indolent PCa in a subject comprising:
obtaining a primary tumor tissue sample from the subject;
assaying the sample to detect methylation status of cytosines located in one or more epigenetic loci selected from Intergenic 1, Intergenic 2, FHAD1, ALKBH5, KLHL8, ATP11A, Intergenic 3, and PI15;
obtaining a value for each selected epigenetic loci based on the assaying;
calculating a β difference; and
determining the sample is metastatic-lethal PCa or indolent PCa based on the β difference(s) thereby distinguishing metastatic-lethal prostate cancer (PCa) from indolent PCa in the subject.

2. A method of claim 1 wherein the cytosines are part of CpG pairs.

3. A method of claim 2 wherein the CpG pairs are within CpG islands.

4. A method of claim 1 wherein the cytosines are within CpG site: cg01135464; CpG site: cg02223001; CpG site: cg02394978; CpG site: cg07166550; CpG site: cg16713292; CpG site: cg21513610; CpG site: cg22501793; and/or CpG site: cg24349665,

5. A method of claim 1 wherein the cytosines are all cytosines within CpG pairs within the selected epigenetic loci.

6. A method of claim 1 wherein the calculating a β difference utilizes a reference level derived from a population of subjects with indolent PCa.

7. A method of claim 6 wherein a positive β difference for Intergenic 1, Intergenic 2, Intergenic 3, and/or PI15 distinguishes the sample as metastatic-lethal PCa.

8. A method of claim 6 wherein a negative β difference for Intergenic 1, Intergenic 2, Intergenic 3, and/or PI15 distinguishes the sample as indolent PCa.

9. A method of claim 6 wherein a negative β difference for FHAD1, ALKBH5, KLHL8, and/or ATP11A distinguishes the sample as metastatic-lethal PCa.

10. A method of claim 6 wherein a positive β difference for FHAD1, ALKBH5, KLHL8, and/or ATP11A distinguishes the sample as indolent PCa.

11. A method of claim 1 wherein calculating a β difference utilizes a reference level derived from a population of subjects with metastatic-lethal PCa.

12. A method of claim 11 wherein a negative β difference for Intergenic 1, Intergenic 2, Intergenic 3, and/or PI15 as compared to the reference level distinguishes the sample as indolent PCa.

13. A method of claim 11 wherein a positive β difference for Intergenic 1, Intergenic 2, Intergenic 3, and/or PI15 distinguishes the sample as metastatic-lethal PCa.

14. A method of claim 11 wherein a positive β difference for FHAD1, ALKBH5, KLHL8, and/or ATP11A distinguishes the sample as indolent PCa.

15. A method of claim 11 wherein a negative β difference for FHAD1, ALKBH5, KLHL8, and/or ATP11A distinguishes the sample as metastatic-lethal PCa.

16. A method of claim 1 wherein the method comprises assaying the sample to detect methylation status of cytosines located in Intergenic 1; Intergenic 2; FHAD1; ALKBH5; KLHL8; ATP11A; Intergenic 3; and PI15.

17. A method of claim 1 wherein the method comprises assaying the sample to detect methylation status of cytosines located in Intergenic 1 and the methylation status of cytosines located in one or more of Intergenic 2; FHAD1; ALKBH5; KLHL8; ATP11A; Intergenic 3; and PI15.

18. A method of claim 1 wherein the method comprises assaying the sample to detect methylation status of cytosines located in Intergenic 2 and the methylation status of cytosines located in one or more of Intergenic 1; FHAD1; ALKBH5; KLHL8; ATP11A; Intergenic 3; and PI15.

19. A method of claim 1 wherein the method comprises assaying the sample to detect methylation status of cytosines located in FHAD1 and the methylation status of cytosines located in one or more of Intergenic 1; Intergenic 2; ALKBH5; KLHL8; ATP11A; Intergenic 3; and PI15.

20. A method of claim 1 wherein the method comprises assaying the sample to detect methylation status of cytosines located in ALKBH5 and the methylation status of cytosines located in one or more of Intergenic 1; Intergenic 2; FHAD1; KLHL8; ATP11A; Intergenic 3; and PI15.

21. A method of claim 1 wherein the method comprises assaying the sample to detect methylation status of cytosines located in KLHL8 and the methylation status of cytosines located in one or more of Intergenic 1; Intergenic 2; FHAD1; ALKBH5; ATP11A; Intergenic 3; and PI15.

22. A method of claim 1 wherein the method comprises assaying the sample to detect methylation status of cytosines located in ATP11A and the methylation status of cytosines located in one or more of Intergenic 1; Intergenic 2; FHAD1; ALKBH5; KLHL8; Intergenic 3; and PI15.

23. A method of claim 1 wherein the method comprises assaying the sample to detect methylation status of cytosines located in Intergenic 3 and the methylation status of cytosines located in one or more of Intergenic 1; Intergenic 2; FHAD1; ALKBH5; KLHL8; ATP11A; and PI15.

24. A method of claim 1 wherein the method comprises assaying the sample to detect methylation status of cytosines located in PI15 and the methylation status of cytosines located in one or more of Intergenic 1; Intergenic 2; FHAD1; ALKBH5; KLHL8; ATP11A; and Intergenic 3.

25. A method of claim 1 wherein the method comprises assaying the sample to detect methylation status of cytosines located in one or more of ALKBH5, FHAD1, KLHL8 and PI15 and wherein the methylation status improves the prognostic determination of a Gleason score.

26. A method of claim 25 wherein the Gleason score is a 4+3=7 or a 3+4=7 Gleason score.

27. A method of claim 25 wherein the Gleason score is a <=6 or a 8-10 Gleason score.

28. A method of claim 1 wherein the sample is a primary PCa tumor sample.

29. A method of claim 25 wherein the improved prognostic determination reduces false negatives.

30. A method of claim 25 wherein the improved prognostic determination reduces false positives.

31. A method of claim 1 wherein distinguishing the PCa as metastatic-lethal directs an aggressive or experimental treatment.

32. A method of claim 1 wherein distinguishing the PCa as indolent directs moderate, minimal, or no treatment.

33. A method of claim 1 wherein the assaying comprises a bisulfite-based methylation assay.

34. A method of claim 1 wherein the assaying comprises pyrosequencing.

35. A method of any of claims 1-34 wherein the β difference is a mean β difference.

36. A kit for distinguishing metastatic-lethal PCa from indolent PCa comprising reagents to detect methylation status of cytosines located in one or more epigenetic loci selected from Intergenic 1, Intergenic 2, FHAD1, ALKBH5, KLHL8, ATP11A, Intergenic 3, and PI15.

37. A kit of claim 36 comprising bisulfate and PCR primers specific for bisulfite-converted DNA.

38. A kit of claim 36 including DNA-fragmenting enzymes.

39. A kit of claim 36 comprising target-specific probes for CpG pairs within any of SEQ ID NOs: 1-5.

40. A kit of claim 36 comprising target-specific probes for CpG pairs within CpG site: cg01135464; CpG site: cg02223001; CpG site: cg02394978; CpG site: cg07166550; CpG site: cg16713292; CpG site: cg21513610; CpG site: cg22501793; and/or CpG site: cg24349665.

41. A kit of claim 36 comprising one or more nucleotides including one or more of SEQ ID NOs: 6-29.

42. A kit of claim 36 comprising a reference level.

43. A kit of claim 42 wherein the reference level is derived from a population of subjects with indolent PCa.

44. A kit of claim 43 wherein up-regulation of methylation status at Intergenic 1, Intergenic 2, Intergenic 3, and/or PI15 as compared to the reference level distinguishes the sample as metastatic-lethal PCa.

45. A kit of claim 43 wherein lack of a statistically-significant difference in methylation status at Intergenic 1, Intergenic 2, Intergenic 3, and/or PI15 as compared to the reference level distinguishes the sample as indolent PCa.

46. A kit of claim 43 wherein down-regulation of methylation status at FHAD1, ALKBH5, KLHL8, and/or ATP11A as compared to the reference level distinguishes the sample as metastatic-lethal PCa.

47. A kit of claim 43 wherein lack of a statistically-significant difference in methylation status at FHAD1, ALKBH5, KLHL8, and/or ATP11A as compared to the reference level distinguishes the sample as indolent PCa.

48. A kit of claim 42 wherein the reference level is derived from a population of subjects with metastatic-lethal PCa.

49. A kit of claim 48 wherein down-regulation of methylation status at Intergenic 1, Intergenic 2, Intergenic 3, and/or PI15 as compared to the reference level distinguishes the sample as indolent PCa.

50. A kit of claim 48 wherein lack of a statistically-significant difference in methylation status at Intergenic 1, Intergenic 2, Intergenic 3, and/or PI15 as compared to the reference level distinguishes the sample as metastatic-lethal PCa.

51. A kit of claim 48 wherein up-regulation of methylation status at FHAD1, ALKBH5, KLHL8, and/or ATP11A as compared to the reference level distinguishes the sample as indolent PCa.

52. A kit of claim 48 wherein lack of a statistically-significant difference in methylation status at FHAD1, ALKBH5, KLHL8, and/or ATP11A as compared to the reference level distinguishes the sample as metastatic-lethal PCa.

53. A kit of claim 36 wherein the kit comprises reagents to detect methylation status of cytosines located in Intergenic 1; Intergenic 2; FHAD1; ALKBH5; KLHL8; ATP11A; Intergenic 3; and PI15.

54. A kit of claim 36 wherein the kit comprises reagents to detect methylation status of cytosines located in Intergenic 1 and methylation status of cytosines located in one or more of Intergenic 2; FHAD1; ALKBH5; KLHL8; ATP11A; Intergenic 3; and PI15.

55. A kit of claim 36 wherein the kit comprises reagents to detect methylation status of cytosines located in Intergenic 2 and methylation status of cytosines located in one or more of Intergenic 1; FHAD1; ALKBH5; KLHL8; ATP11A; Intergenic 3; and PI15.

56. A kit of claim 36 wherein the kit comprises reagents to detect methylation status of cytosines located in FHAD1 and methylation status of cytosines located in one or more of Intergenic 1; Intergenic 2; ALKBH5; KLHL8; ATP11A; Intergenic 3; and PI15.

57. A kit of claim 36 wherein the kit comprises reagents to detect methylation status of cytosines located in ALKBH5 and methylation status of cytosines located in one or more of Intergenic 1; Intergenic 2; FHAD1; KLHL8; ATP11A; Intergenic 3; and PI15.

58. A kit of claim 36 wherein the kit comprises reagents to detect methylation status of cytosines located in KLHL8 and methylation status of cytosines located in one or more of Intergenic 1; Intergenic 2; FHAD1; ALKBH5; ATP11A; Intergenic 3; and PI15.

59. A kit of claim 36 wherein the kit comprises reagents to detect methylation status of cytosines located in ATP11A and methylation status of cytosines located in one or more of Intergenic 1; Intergenic 2; FHAD1; ALKBH5; KLHL8; Intergenic 3; and PI15.

60. A kit of claim 36 wherein the kit comprises reagents to detect methylation status of cytosines located in Intergenic 3 and methylation status of cytosines located in one or more of Intergenic 1; Intergenic 2; FHAD1; ALKBH5; KLHL8; ATP11A; and PI15.

61. A kit of claim 36 wherein the kit comprises reagents to detect methylation status of cytosines located in PI15 and methylation status of cytosines located in one or more of Intergenic 1; Intergenic 2; FHAD1; ALKBH5; KLHL8; ATP11A; and Intergenic 3.

62. A kit of claim 36 comprising reagents to perform a bisulfite-based methylation assay.

63. A kit of claim 36 comprising reagents to perform pyrosequencing.

64. A kit of claim 36 comprising probes and silicon based arrays to perform a methylation assay.

65. A kit of claim 64 wherein the probes comprise beads.

66. A kit of claim 65 wherein the beads are configured to allow a detectable label to be bound.

67. A kit of claim 36 comprising proteins and/or nucleotide sequences that bind to one or more proteins encoded by, and/or one or more nucleotide sequences corresponding to, one or more of Intergenic 1; Intergenic 2; FHAD1; ALKBH5; KLHL8; ATP11A; Intergenic 3; and PI15.

68. A kit of claim 36 comprising a DNA nucleotide sequence and/or a RNA nucleotide sequence.

69. A kit of claim 67 wherein the proteins comprise antibodies, epitopes, or mimitopes.

70. A kit of claim 36 comprising a detectable label.

71. A kit of claim 70 wherein the detectable label is a radioactive isotope, enzyme, dye, fluorescent dye, magnetic bead, or biotin.

72. A method to improve the prognostic determination of a subject's Gleason score comprising distinguishing metastatic-lethal prostate cancer (PCa) from indolent PCa in the subject by:
obtaining a primary tumor tissue sample from the subject;
assaying the sample to detect methylation status of cytosines located in one or more epigenetic loci selected from Intergenic 1, Intergenic 2, FHAD1, ALKBH5, KLHL8, ATP11A, Intergenic 3, and PI15;
obtaining a value for each selected epigenetic loci based on the assaying;
calculating a β difference; and
determining the sample is metastatic-lethal PCa or indolent PCa based on the β difference(s) thereby distinguishing metastatic-lethal prostate cancer (PCa) from indolent PCa in the subject and improve the prognostic determination of the subject's Gleason score.

73. A method of claim 72 wherein the cytosines are part of CpG pairs.

74. A method of claim 73 wherein the CpG pairs are within CpG islands.

75. A method of claim 72 wherein the cytosines are within CpG site: cg01135464; CpG site: cg02223001; CpG site: cg02394978; CpG site: cg07166550; CpG site: cg16713292; CpG site: cg21513610; CpG site: cg22501793; and/or CpG site: cg24349665,

76. A method of claim 72 wherein the cytosines are all cytosines within CpG pairs within the selected epigenetic loci.

77. A method of claim 72 wherein the calculating a β difference utilizes a reference level derived from a population of subjects with indolent PCa.

78. A method of claim 77 wherein a positive β difference for Intergenic 1, Intergenic 2, Intergenic 3, and/or PI15 distinguishes the sample as metastatic-lethal PCa.

79. A method of claim 77 wherein an equal or negative β difference for Intergenic 1, Intergenic 2, Intergenic 3, and/or PI15 distinguishes the sample as indolent PCa.

80. A method of claim 77 wherein a negative β difference for FHAD1, ALKBH5, KLHL8, and/or ATP11A distinguishes the sample as metastatic-lethal PCa.

81. A method of claim 77 wherein an equal or positive β difference for FHAD1, ALKBH5, KLHL8, and/or ATP11A distinguishes the sample as indolent PCa.

82. A method of claim 72 wherein the calculating a β difference utilizes a reference level derived from a population of subjects with metastatic-lethal PCa.

83. A method of claim 82 wherein a negative β difference for Intergenic 1, Intergenic 2, Intergenic 3, and/or PI15 as compared to the reference level distinguishes the sample as indolent PCa.

84. A method of claim 82 wherein an equal or positive β difference for Intergenic 1, Intergenic 2, Intergenic 3, and/or PI15 distinguishes the sample as metastatic-lethal PCa.

85. A method of claim 82 wherein a positive β difference for FHAD1, ALKBH5, KLHL8, and/or ATP11A distinguishes the sample as indolent PCa.

86. A method of claim 82 wherein an equal or negative β difference for FHAD1, ALKBH5, KLHL8, and/or ATP11A distinguishes the sample as metastatic-lethal PCa.

87. A method of claim 72 wherein the method comprises assaying the sample to detect methylation status of cytosines located in Intergenic 1; Intergenic 2; FHAD1; ALKBH5; KLHL8; ATP11A; Intergenic 3; and PI15.

88. A method of claim 72 wherein the method comprises assaying the sample to detect methylation status of cytosines located in Intergenic 1 and the methylation status of cytosines located in one or more of Intergenic 2; FHAD1; ALKBH5; KLHL8; ATP11A; Intergenic 3; and PI15.

89. A method of claim 72 wherein the method comprises assaying the sample to detect methylation status of cytosines located in Intergenic 2 and the methylation status of cytosines located in one or more of Intergenic 1; FHAD1; ALKBH5; KLHL8; ATP11A; Intergenic 3; and PI15.

90. A method of claim 72 wherein the method comprises assaying the sample to detect methylation status of cytosines located in FHAD1 and the methylation status of cytosines located in one or more of Intergenic 1; Intergenic 2; ALKBH5; KLHL8; ATP11A; Intergenic 3; and PI15.

91. A method of claim 72 wherein the method comprises assaying the sample to detect methylation status of cytosines located in ALKBH5 and the methylation status of cytosines located in one or more of Intergenic 1; Intergenic 2; FHAD1; KLHL8; ATP11A; Intergenic 3; and PI15.

92. A method of claim 72 wherein the method comprises assaying the sample to detect methylation status of cytosines located in KLHL8 and the methylation status of cytosines located in one or more of Intergenic 1; Intergenic 2; FHAD1; ALKBH5; ATP11A; Intergenic 3; and PI15.

93. A method of claim 72 wherein the method comprises assaying the sample to detect methylation status of cytosines located in ATP11A and the methylation status of cytosines located in one or more of Intergenic 1; Intergenic 2; FHAD1; ALKBH5; KLHL8; Intergenic 3; and PI15.

94. A method of claim 72 wherein the method comprises assaying the sample to detect methylation status of cytosines located in Intergenic 3 and the methylation status of cytosines located in one or more of Intergenic 1; Intergenic 2; FHAD1; ALKBH5; KLHL8; ATP11A; and PI15.

95. A method of claim 72 wherein the method comprises assaying the sample to detect methylation status of cytosines located in PI15 and the methylation status of cytosines located in one or more of Intergenic 1; Intergenic 2; FHAD1; ALKBH5; KLHL8; ATP11A; and Intergenic 3.

96. A method of claim 72 wherein the Gleason score is a 3+4=7 or a 4+3=7 Gleason score.

97. A method of claim 72 wherein the Gleason score is a <=6 or a 8-10 Gleason score.

98. A method of claim 72 wherein the sample is a primary PCa tumor sample.

99. A method of claim 72 wherein the improved prognostic determination reduces false negatives.

100. A method of claim 72 wherein the improved prognostic determination reduces false positives.

101. A method of claim 72 wherein the assaying comprises a bisulfite-based methylation assay.

102. A method of claim 72 wherein the assaying comprises pyrosequencing.

103. A method of any of claims 72-102 wherein the β difference is a mean β difference.
